(19) United States
(12) Patent Application Publication
(10) Pub. No.: US 2018/0237734 A1
(43) Pub. Date: Aug. 23, 2018

(54) METHODS AND BIOREACTORS FOR MICROBIAL DIGESTION USING IMMOBILIZED BIOFILMS

(71) Applicant: Dong Energy Thermal Power A/S, Fredericia (DK)

(72) Inventor: Bjarne Uller, Odense SØ (DK)

(73) Assignee: Dong Energy Thermal Power A/S, Fredericia (DK)

(21) Appl. No.: 15/514,911

(22) PCT Filed: Sep. 30, 2015
(86) PCT No.: PCT/EP2015/072650

§ 371 Date: Mar. 28, 2017

Related U.S. Application Data
(60) Provisional application No. 62/057,265, filed on Sep. 30, 2014.

(51) Int. Cl. C12M 1/107; C12M 1/00; B01J 8/02; B01J 8/06; C02F 3/28

(52) U.S. Cl. CPC C12M 21/04; C12M 1/107; C12M 27/20; B01J 8/0292; B01J 2208/00876; C02F 3/2806; C02F 3/2873; C02F 2203/006; B01J 8/06

(57) ABSTRACT

The invention relates to methods, devices and inserts for reactors for microbial and anaerobic digestion. In particular, the invention relates to reactors comprising inserts for biofilms, such as methane-producing biofilms, immobilized on a carrier matrix.

### FIELD OF THE INVENTION

The invention relates generally to methods and reactors for microbial digestion and specifically to methods and reactors comprising an insert comprising a biofilm immobilized on a carrier matrix.

The invention also relates to methods and reactors for anaerobic digestion and specifically to methods and reactors in which a methane-producing biofilm is immobilized on a carrier matrix having fixed orientation.

### BACKGROUND OF THE INVENTION

A wide variety of industries produce waste streams that require further biological processing to recover clean water content and “energy from waste.” In many cases such as, for example, with distillery vinasse, liquefied organic components of municipal solid waste (MSW), and wastes from abattoirs, restaurants, dairy processing, and tanneries, these waste streams contain a high level of total solids, typically greater than 7% by weight. In the absence of additional processing steps, these waste streams inevitably become acidic due to spontaneous fermentation by ubiquitous bacteria. It is clearly advantageous that these waste streams be processed on the site where they are produced. However, in the case of anaerobic digestion to biomethane, these waste streams have proved problematic, where conventional technology based on continuously stirred tank reactors (CSTR) was applied.

In CSTR systems, the microbiological consortium that metabolizes feed streams to methane, carbon dioxide and ammonia, is free-floating in solution, typically in flocs. The critical methanogenic Archaea are slow-reproducing and highly sensitive to external conditions. This renders CSTR systems notoriously prone to the phenomenon of substrate inhibition, or volatile fatty acid (VFA) toxicity. In response to high VFA levels in CSTR systems, Archaea stop reproducing and enter a state of metabolic dormancy. In order to avoid “overfeeding” the reactor, which results in VFA toxicity, CSTR systems typically require elaborate process controls and long digester retention times, typically 15 days or longer. Acidic and high-solids waste streams have proved unmanageable in CSTR systems due to problems associated with VFA toxicity—bursts of overproduction or drops in pH to levels at which methanogens cease to metabolize gives rise to accumulation of VFA.

CSTR systems are also notoriously prone to salt toxicity. The precise mechanisms of salt toxicity remain obscure and likely differ depending on the metal ion involved—different cations when mixed are reported to exert, alternatively, antagonistic or synergistic toxic effects. (For review see Chen et. al 2008).

Acidic waste streams are especially troublesome in CSTR systems. Control of pH in anaerobic digestion is a critical problem with complex dependencies on feed stream properties and on the buffering capacity of the reactor liquid volume at any given moment. (For review see Anderson and Yang 1992). Increasing organic load on the reactor (i.e, processing a high solids feed at shorter retention time) increases the requirement for buffering capacity. The inherent requirement for buffering capacity is further increased, where the feed stream is, itself, acidic. In order for pH in a CSTR reactor not to fall beneath 6.5, at which level most methanogens cease to metabolize, the feedstock stream must typically be subject to pH adjustment. By far the most inexpensive means for chemical pH adjustment is sodium hydroxide. However the resulting increased sodium content of the feed stream is itself potentially toxic in a CSTR system. Ammonium bicarbonate is possibly preferable as a means for pH adjustment, but then the resulting increased ammonia content is also potentially toxic, presumably for different reasons (see Chen et al. 2008).

Salt toxicity and also VFA toxicity due to “overfeeding” and also VFA toxicity due to acidity problems with the feed stream typically result in a “shutdown” event in conventional CSTR systems. Even where the underlying toxicity is theoretically reversible, such as is likely the case with VFA toxicity, the toxic event nevertheless leads to production shutdowns. This is presumably largely due to seizure of methanogen reproduction and subsequent washout of productive microbes. A conventional CSTR reactor cannot recover simply by dilution of the substrate, since this dilutes the productive microbial community as well. Recovery from toxic events in CSTR systems typically requires discarding the digester content and re-seeding with microbiological cultures, which in turn leads to commercially disastrous, long production stops.

As a consequence of these problems, anaerobic digestion of many of these problematic industrial waste streams has been achieved commercially only by mixing with more manageable, lower solids substrates such as manure—usually at remote processing locations. While this provides functional recovery of water and “waste energy,” it is a far less convenient solution than dedicated on-site processing.

In contrast with conventional CSTR systems, so-called “fixed film” bioreactors are well known to provide reduced sensitivity to toxicity in general. In fixed film reactors, the microbiological consortium is assimilated within a biofilm. Bacteria and archaea are interspersed within the biofilm in a matrix comprising exopolymeric substances (EPS) produced by bacteria as well as a heterogeneous mixture of other biological macromolecules. A typical biofilm in an anaerobic digestion system comprises an outer surface that acts as a diffusion barrier. The film can vary in thickness from very thin, on the order of 200 um (see e.g. Mahendran et al. 2012), to moderately “thick,” between 2-5 mm (see e.g. Hickey et al. 1991). The relative proportion of methanogenic Archaea to bacteria in CSTR systems is typically between 10-25% (See e.g. Leclerc et al. 2004; and see Regueiro et al. 2012. In contrast, in fixed film systems subject to high-VFA loading, methanogenic Archaea can predominate over bacteria. (See e.g. Hickey et al. 1991). In some fixed film systems, the biofilm is formed on an immobilization media. The chemical nature of this support material can affect the properties of biofilms formed, most notably thickness and density of productive biomass. See Habouzit et al. 2014 and see Adu-Gyamfi et al. 2012

At the very least, fixed film systems are more resilient than CSTR systems simply because the productive microbiological consortium cannot be “washed out” of the reactor. Thus, to the extent that a toxic event is reversible, toxic levels of sodium, ammonia or VFA can simply be washed out of a fixed film reactor, with little or no loss of productive biomass and with comparatively rapid recovery of production.

But there are likely many others reason why fixed-film systems are more robust to particular toxic challenges than CSTR systems. It is clearly possible that the arrangement of mutually interdependent bacteria and archaea in close physical proximity, within a film, and the functional properties of the film itself could render fixed film systems inherently less sensitive to various forms of toxicity. For example, fixed cells might undergo physiological adaptations due to high local concentrations of nutrient substrates or enzymes within the biofilm. Alternatively, and not mutually exclusively, cells within a biofilm may experience a reduced effective concentration of toxic substances relative to that in the bulk aqueous phase. Both pH and metabolite concentrations are known to vary in biofilms in such manner as to produce a depth-dependent gradient (See Allen et al. 1999; Arcand et al. 1994; Suidan et al. 1984; Suidan et al. 1994; Van Whey et al. 2011; Annachatre and Khanna 1990). Furthermore, selective effects of the EPS matrix itself could exert a protective effect. Bacterial exopolymers are, for example, known to bind sodium, which might serve to ameliorate salt toxicity (see e.g. WO2007044439 “Microbial exopolymers useful for water demineralization” and see Vivanco et al. 2006).

A wide variety of different fixed film biomethane systems have been reported. (For a comprehensive review, see Tauseef et al. 2013). These systems have been presented under a variety of different names, such as “anaerobic filter” (AF), “downflow stationary fixed filter” (DSFF), “upflow anaerobic sludge blanket” (UASB), “anaerobic fluidized bed” (AFSBR), “anaerobic sequence batch reactor” (ASBR), “anaerobic baffle reactor” (AFBR), “anaerobic fixed bed” (AFFB), and so on. However, notwithstanding the wide variety of reported reactor configurations, fixed film biomethane systems essentially break down into three general categories:

In a first category of fixed film systems, biofilm is effectively suspended in solution, i.e., free floating in the reactor tank. These “suspended” fixed film systems include reactors in which the biofilm has formed itself within free standing granules or, alternatively, on “mobile” immobilization media. Granular sludge systems can be arranged in a variety of ways. For example, sludge granules may be augered (see e.g. Chen et al. 2010) or allowed to float as a “sludge blanket” (see e.g. Mohan et al. 2007) or compartmentalized (see e.g. Ji et al. 2012) or driven through a system of baffles (see e.g. Alkarimiah et al. 2011) or used as a “hybrid sludge blanket” having a filter on the upper layer to prevent outflow loss of granules (see e.g. Banu and Kaliappan 2007) or in some other configuration. Similarly, a wide variety of different biofilm immobilization media can be used which is then allowed to float freely in a reactor tank, for example, specialized polyethylene carriers with blades providing surface area (Chai et al. 2014), pieces of polyvinylchloride (PVC) pipe (Pradeep et al. 2014), or latex beads (Wu et al. 2003).

In a second category of fixed film systems, the biofilm is formed on immobilization media which is employed in the reactor with random orientation in a stationary bed. For example random orientation fixed bed systems have been reported using immobilization media such as synthetic nylon pads (Deshpande et al. 2012), nylon fibers (Meesap et al. 2012), corrugated plastic rings (Martin et al. 2010), silica beads (Michaud et al. 2005), polypropylene rings (Austermann-Haun et al. 1994), or clay beads (Wildenauer and Winter 1985), which are used in random orientation to form a packed bed.

In a third category of fixed film systems, the biofilm is formed on immobilization media which is employed in the reactor with non-random orientation to form a fixed bed through which fluid flow can be more carefully controlled. These fixed orientation, fixed bed systems have been viewed as a means for extending the range of tolerance to higher suspended solids content in the feed stream relative to random bed systems. (See Kennedy and van den Berg 1982 [18 g/L]; del Pozo et al. 2000 [1 g/L suspended solids]; Escudie et al. 2005 [2-3 g/L suspended COD]).

The primary problem that has plagued fixed film systems when attempting to operate at high organic load has been susceptibility to “clogging” or blockage of productive exchange at the biofilm surface. The clogging problem arises from a variety of different sources. At the simplest level, clogging is simply a function of suspended solids content in the feed stream. Thus, except with feed streams that have been subject to an additional precipitation step such as, for example, electrocoagulation (see e.g. Deshpande et al. 2012), high total solids typically imparts higher suspended solids. Random orientation fixed bed systems and granule systems typically provide extremely fast and effective processing of feed streams having lower content of chemical oxygen demand (COD) (<30 g/L) or suspended solids (<3% w/w). For example, for random orientation fixed bed systems reviewed by Tauseef et al. 2013, the highest reported organic load rate (OLR) that could be sustained with at least 70% COD removal was 18 g/L digester volume/day, with an overall average of 6.8 g/L digester volume/day. The highest reported biogas production rates sustainable with at least 70% COD removal was 4.2 L/L digester volume/day, with an overall average of 2.4 L/L digester volume day. But while effective with low solids feed streams, these systems inevitably run slower and less effectively as COD content of the feed stream increases, eventually becoming clogged after long periods of operation at high solids loading, or at some terminal tolerance level of solids content. Fixed orientation fixed bed systems, in contrast, typically can tolerate a higher COD content in the feed stream and maintain a higher organic load at some defined level of COD removal (for example 70% or greater). (See del Pozo et al 2000; Escudie et al. 2005; Kennedy and van den Berg 1982).

A second factor contributing to clogging in fixed film systems is the tendency of these systems to experience “channelling” effects in fluid stream flows through and around the immobilization media or granules. These effects are particularly pronounced in granule and “suspended” carrier systems and also in random orientation fixed bed systems, where microscopic non-homogeneous flow patterns result in internal bypass flows and formation of dead volumes. But this tendency for “channelling” also occurs in fixed orientation fixed bed systems, albeit at a diminished level. “Channelling” effects in fixed orientation fixed bed systems create a kind of feed-forward cascade of clogging: “Channelling” results in regional accumulation of attached solids at particular locations in the flow pattern through the support media. (See e.g. Hall 1982 [internal—p. 393, col 1, point 3]; and see e.g. del Pozo et al. 2000 [internal—p. 221]. This regional accumulation of attached solids in turn further exacerbates the tendency for “channelling” and promotes additional accumulation of attached solids at other locations.

Flow patterns may be referred to as flow path within the meaning of this application.

A third factor contributing to clogging in fixed film systems, which is considered the most important factor in fixed bed systems (Escudie et al. 2011), is the growth of the film itself and the gradual accumulation of suspended biomass in the form of flocs or detached segments of biofilm. (See Rajeshwari et al. 2000; Lima et al. 2005). The formation and maintenance of biofilm is generally believed to be described by a dynamic equilibrium between attachment and detachment of film segments (See van Loosdrecht et al. 1995), which is in turn influenced by an equilibrium between readily precipitable flocs and precipitation-resilient bacterial colloids. (See Albizuri et al. 2014). The balance between cohesion and detachment in biofilm maintenance is strongly affected by shear forces, which promote detachment of biofilm segments. These detached segments in turn greatly increase the risk of clogging. (van Loosdrecht 1995; Escudie 2011).

In fixed orientation fixed bed systems of the prior art, clogging has eventually become a problem during long term use. For example, in the largest scale and most successful such system reported to date, over the course of 7 years' operation, the immobilization matrix comprising tubular channels gradually became occluded by expansion of the biofilm, i.e., attachment of suspended biomass. (Escudie et al 2011). In this upflow system, winery vinasse having between 20-40 g/L COD and between 2-3 g/L suspended solids was continuously processed with recirculation of effluent. During the course of operations, so much biomass accumulated on the immobilization matrix that the reactor eventually lost 75% of its functional internal volume.

Fixed orientation, fixed bed systems have, accordingly, not previously been considered commercially feasible for processing high solids waste streams, much less streams having high suspended solids. Conventional CSTR systems continue to predominate in the industry in this context, despite their wellknown disadvantages.

We present here, amongst other things, methods of producing e.g. biomethane and systems for practicing these methods which can be used to retrofit conventional commercial CSTR tanks into robust and efficient fixed orientation, fixed bed bioreactors for processing high solids waste streams. The advantage of retrofitting, adapting or modifying an existing CSTR to a Fast Anaerboic Digestion (FAD) as disclosed herein comprise e.g. a more robust and faster process, and a dramatic increase in productivity due to the higher flow rates. Furthermore, down time is reduced, and start-up time, e.g. after system maintenance, is also significantly reduced. Furthermore, a greater flexibility towards substrate variations is provided.

Suspended solids, notably including suspended biomass (such as or including detached biofilm segments and flocs), are gently precipitated within sedimentation zones that exist beneath neighbouring chambers of a compartmentalized reactor. Vertical flow paths ensure that precipitating particles will be directed into a sedimentation zone. The avoidance of agitation in favour of gentle, backflow mixing ensures that suspended particles will indeed precipitate in the sedimentation zones. A downward, low-shear plug flow, which imparts minimal risk of channelling, is directed through a plurality of tubular immobilization carriers, contained within a single chamber of the reactor. This downward flow is then directed onward into a sedimentation zone situated beneath the carriers. There within the sedimentation zone, the vertical direction of flow is forced to change into an upward, low-shear plug flow through tubular immobilization carriers contained within a succeeding chamber. As the flow proceeds through other chambers of the compartmentalized reactor, the vertical direction is forced to change between each successive chamber, thereby achieving a gentle backflow mixing both within sedimentation zones situated beneath the immobilization carriers, as well as within head space regions situated above the carriers. Flow velocity through the system is determined by the effluent recirculation rate, by the dimensions of the reactor tank and by the number and dimensions of individual compartments within the reactor. A reactor of the invention can be fitted with means for periodic removal of undissolved solids from sedimentation zones.

Ironically and remarkably, the high solids content of feed streams that has proved troublesome in prior art systems is actively believed to be beneficial in systems of the invention. High COD content of the feed stream permits maintenance of extremely high biogas flows, provided that low hydraulic retention times (HRT) are maintained. Without wishing to be bound by theory, we consider that these very high biogas flows, emerging from within the biofilm, impart a protective effect whereby biofilm performance is improved and the tendency for clogging greatly reduced.

The emergence of tiny bubbles at the biofilm/feed stream interface can be expected to impart some degree of microscopic shear force and to promote local back-mixing, notwithstanding the plug-flow character of the feed stream flow. Indeed, biogas production in fixed orientation fixed bed systems has previously been suggested to minimize the risk of channelling (see Hall 1982) and has previously been shown to promote mixing (see Escudie et al. 2005).

It can be shown that biogas bubbles rise to the head space of systems of the invention by travelling along the surface of the biofilm, presumably involving both coalescence and cavitation events. These are conditions that, at high gas production rates, should encourage detachment of biofilm segments but discourage re-attachment. Detached biofilm segments according to this theory simply precipitate harmlessly in sedimentation zones. This in turn effectively shifts the equilibrium between biofilm growth and detachment in favour of a thin, smooth, dense, and highly productive film (see Loosrecht et al. 1995). This proposed effect could possibly bear some similarity on a microscopic level to the macroscopic effect observed in sludge blanket systems whereby sustained constant high flow rates of re-circulated biogas provide good control of biomass accumulation, promoting a thin, productive biofilm. (See Michaud et al. 2003).

Maintenance of low retention time also ensures that colloidal suspended biomass will be rapidly flushed from the system, further reducing the risk of excessive biomass accumulation within the immobilization matrix.

By practicing methods of the invention, diverse acidic feed streams having high solids content can be processed by anaerobic digestion, without requirement for pH adjustment, but with high speed, high methane yield, and virtually complete immunity from VFA toxicity arising from “overfeeding.”

In the context of the present invention, the term “feedstock” or “substrate” means a cellulosic, hemicellulosic, lignocellulosic or starch containing biomass.

The term “biomass” means any biomass, such as waste, sewage, manure, wheat straw, corn stover, sugar cane bagasse, sweet sorghum bagasse, or empty fruit bunches.

In this context, the term “waste” means any kind of waste having an organic content, such as municipal solid waste (MSW), industrial waste, animal waste or plant waste.

In the context of the present invention, the term “hydrothermal pre-treatment” refers to the use of water, either as hot liquid, vapor steam or pressurized steam comprising high temperature liquid or steam or both, to “cook” biomass, at temperatures of 120° C. or higher, either with or without addition of acids or other chemicals.

In the context of the present invention, the term “anaerobic digestion” refers to microbial fermentation under controlled aeration conditions, e.g. in absence or very limited amount of oxygen gas in which methane gas is produced. Methane gas is produced to the extent that the concentration of metabolically generated dissolved methane in the aqueous phase of the fermentation mixture within the “anaerobic digestion” is saturating at the conditions used and methane gas is emitted from the system.

The term “aerobic digestion” refers to microbial fermentation conducted under aerated conditions.

In the context of the present invention, the term “COD or Chemical Oxygen Demand” means the amount of oxygen which is needed for the oxidation of all organic substances in water in g/L and hence is a measure for the organic content of the feedstock or biomass.

Thus, the above described object and several other objects are intended to be obtained in a first aspect of the invention by providing a biofilm carrier suitable for biofilm growth upon exposure to a flow of fluid containing biofilm precursors, the biofilm carrier comprising a three dimensional structure having at least one surface comprising cavities and protrusions thereby providing a rough surface.

Biofilm carrier may be referred to as biofilm support, biofilm matrix immobilizer or carrier matrix.

The protrusions may be extending out of the at least one surface between 0.1 and 10 mm. The cavities or indentations may be in the area between 0.1 and 5 mm underneath the at least one surface.

The at least one surface is a rough surface is preferably being a rough surface, i.e. a surface that is not smooth. The at least one surface may have a rough surface area (Rₐ) between 0.1 and 10 mm, such as between 1 and 9 mm, for example between 2 and 8 mm. Preferably Rₐ may be between 3 and 6 mm. The at least one surface may have a minimum valley depth Rᵥ between 0.5 and 1.5 mm, such as 1 mm. The at least one surface may have a minimum peak depth Rₚ between 1 and 2 mm, such as 2 mm.

The specific surface roughness has the advantage of allowing regrowth of the biofilm that has been at least partially washed out. During operation, it may occur that biofilm segments or flocs detach from the biofilm carrier. Regrowth of biofilm may not be straightforward as the at least one surface is exposed to continuous fluid flow, thus not allowing for optimal regrowth condition. The presence on the surface of elements, such as cavities or protrusions that are less exposed to fluid flow has thus the advantage of allowing for biofilm regrowth. Indeed, biofilm regrowth may start within the elements that are less exposed to the fluid flow.

The specific surface roughness has also the advantage of increasing the surface area available for biofilm growth and thus increasing the surface available for biofilm digestion of the feedstock introduced in contact with the biofilm carrier.

In some embodiments the three dimensional structure comprises openings, such as holes throughout the at least one surface.

For example, in the biofilm carrier, the three dimensional structure may be a tubular porous three dimensional structure. The porous may be open porous, i.e. a porous having at least an open end.

In other embodiments, the three dimensional structure is or comprises a threaded structure. For example, the three dimensional structure is or comprises an open threaded structure.

A three dimensional open threaded structure may be made of two or more filaments twisted and attached together.

The advantage of having three dimensional structure comprising openings, whether these are open porous, throughout holes or an open threaded structure, is that the contact with the fluid flow may occur from both sides of the three dimensional structure where the flow of the fluid may have different characteristics. For example, fluid flow may have different speed, different quality, e.g. different biogas producing potential, different temperature, just to name some. These different characteristics may allow preferential biofilm growth starting from one specific side of the three dimensional structure. For example, a first side of three dimensional structure may be exposed to a fluid flow having a higher speed than the one on a second side. Thus, in case of biofilm detachment, biofilm growth on the first side may not occur while biofilm growth on the second side may occur, eventually extending towards the first side through the opening.

Thus, a three dimensional structure comprising openings allows for faster regrowth of biofilm in case of partial or total detachment of the biofilm from the biofilm carrier.

In some embodiments, the biofilm carrier comprises a biofilm. The biofilm may be a biofilm comprising one or more different microorganisms adapted to aerobic or anaerobic digestion/fermentation This embodiment has the advantage that biofilms grown on the biofilm carrier may be thus transported away from the growth environment, such as a tank reactor, and located in other apparatus, reactor or inserts for modifying reactors so as to be used for producing gas or other products. A biofilm carrier actually carrying the biofilm may be treated so as to maintain its characteristics during transport, e. g. may be thermally treated, such as eventually frozen or protected, such as coated with a protective layer. Biofilm carriers comprising the biofilm may be very robust, i.e. may be transported as such, without e.g. the need of a thermal treatment, without a protective layer and/or protective or controlled atmosphere.

In a second aspect, the invention relates to an insert comprising one or more means for restraining the flow of a fluid, such as one or more baffles defining at least two open compartments, the one or more baffles comprising one or more open edges, thereby when inserted into a tank reactor and when the tank reactor is in operation, the one or more open edges define an underflow or an overflow aperture thus forcing a fluid to flow upwardly or downwardly across the underflow or the overflow aperture.

The insert is suitable for modifying a tank reactor.

The insert has the advantage that, when inserted into a tank reactor and when the tank reactor is in operation, the insert allows for restraining and directing the flow of a fluid with a very low level of maintenance as no mechanical moving parts are present and the one or more baffles are already fixed in the desired position without needing further adjustments. A further advantage is that energy usage or electrical consumption is minimized though the use of the insert.

A even further advantage of the insert is that it is able to restrain and direct a flow of fluid through the system avoiding clogging. The insert defines open compartments allowing for precipitation or deposition of solids suspended into the fluid out of the flow path, i.e. towards and onto the bottom of the open compartments.

In some embodiments, the at least two compartments further comprise a continuous closed side wall surrounding the one or more baffles, wherein the one or more open edges are displaced in respect to a height of the continuous closed side wall.

The one or more open edges are displaced, i. e. staggered or shifted, in respect to a height of the continuous closed side wall, i.e. in respect to an open edge of the continuously closed side wall.

This specific positioning of the open edges ensures the desired vertical zigzag flow, alternating upwardly vertical flow and downwardly vertical flow of a fluid passing through the insert.

In some further embodiments, the one or more baffles are fastened to the continuous closed side wall.

The insert may comprises only one baffle fasten to the continuous closed side wall.

In some embodiments, the continuous closed side wall may be the side wall of the reactor in which the insert is inserted and installed. In this case the insert may comprise simply the one or more baffles fastened, e. g. by means of welding, to the continuous closed side wall of the tank reactor in which the insert is inserted.

In some other embodiments, the one or more baffles are removably attached, i.e. attached in a way that allows for removal, to the continuously closed side wall.

In some other embodiments, the continuous closed side wall is an element of the insert and not of the tank reactor in which the insert may be inserted.

In some embodiments, the continuous closed side wall is a curved wall.

The presence of curved continuous closed side wall has the advantage that the insert can be easily adapted to be inserted in most of the tank reactor currently available, which have at least one curved wall.

In some further embodiments, the one or more baffles are a plurality of baffles and the at least two open compartments are a plurality of open compartments.

In some embodiments, N amount of baffles defines N+1 open compartments, wherein N is a number higher than 1.

In some embodiments, the one or more open edges of the plurality of baffles are displaced in respect to each other, thereby when inserted into a tank reactor and when the tank reactor is in operation the one or more open edges define a plurality of underflow and overflow apertures, thus forcing a fluid to flow from an underflow aperture of a first compartment upwardly towards an overflow aperture of a second subsequent compartment and downwardly towards an underflow aperture of a third subsequent compartment.

In general, when the insert comprises a plurality of compartments, the disposition of underflow and overflow apertures is such that it forces a fluid to flow from an underflow aperture of open compartment M upwardly towards an overflow aperture of subsequent open compartment M+1 and subsequently downwardly towards an underflow aperture of a subsequent open compartment M+2, wherein M is a number higher than 1.

The one or more open edges of the plurality of baffles are displaced, i. e. staggered or shifted, in respect to each other, i.e. the one or more open edges are facing each other at a different level; e. g. at different heights, in respect to the central horizontal plane of the insert.

For example, an open edge of a first baffle facing an open edge of a second baffle may be located at a different height, in respect to the central horizontal plane of the insert so as to ensure the desired vertical zigzag flow, alternating upwardly vertical flow and downwardly vertical flow of a fluid passing through the open compartments of the insert.

In some embodiments the plurality of baffles are interconnected baffles.

Baffles may be interconnected, i. e. connected with each other, by, for example sharing a wall or an edge. In some other embodiments, baffles may intersect each other having different degree of overlap.

In some further embodiments, the one or more of the at least two open compartments defines one or more sections of the insert.

The insert may thus be divided in a plurality of sections comprising a plurality of open compartments. For example, a cylindrical insert may comprises 4 quarter-cylinder sections having substantially equivalent cross sectional area and having a plurality of compartments defined by a plurality of baffles. An outer section may comprise a curved outer wall that defines together with the baffles trapezoidal open compartments having one curved surface formed by the inner surface of the curved outer wall and another curved surface formed by the outer surface of a curved inner wall of an inner section.

This configuration allows for optimal fitting of the insert in currently available reaction tanks.

The one or more sections may thus be external, i. e. an outer section located at the periphery of the insert, or internal, i.e. an inner section located in the centre of the insert.

In some embodiments, the one or more baffles and/or the continuous closed side wall are/is made from a corrosion resistant and liquid impermeable material.

This allow the insert to be used in the harsh environment present during digestion, such as anaerobic digestion.

In some further embodiments, the insert according to the second aspect of the invention further comprises means for supporting biofilms located in the at least two open compartments.

For example, means for supporting biofilms may be biofilm supports, biofilm carriers or other means for immobilizing biofilms on a substrate.

In some embodiments, the means for supporting biofilms are a plurality of biofilm carriers according to the first aspect of the invention.

In some embodiments the insert according to the second aspect of the invention define a preferential vertical path along and inside the biofilm carriers, thereby when inserted into a tank reactor and when the tank reactor is in operation with a fluid flow substantially parallel to the biofilm formation. This has the main advantage of avoiding clogging.

In a third aspect, the invention relates to a bioreactor comprising: a container, such as a tank reactor, having one or more side walls and a bottom wall having an internal surface and a bottom opening; at least two removable, i.e. removably attached, open compartments located inside the container; at least one overflow aperture or underflow aperture between the at least two removable compartments. Thereby, when the bioreactor is in operation a fluid flows between the at least two removable compartments downwardly towards the at least one underflow aperture or upwardly towards the at least one overflow aperture.

In some embodiments, the bioreactor according to the third aspect of the invention comprises the insert according to second aspect of the invention, wherein the at least two removable open compartments are the at least two open compartments defined by the one or more baffles of the insert and wherein the at least one overflow aperture or underflow aperture are the underflow aperture or the overflow aperture defined by the one or more open edges.

In some embodiments, the one or more side walls of the container are the continuous closed side wall of an insert according to the second aspect of the invention.

The side walls of the reactor together with the one or more baffles may thus define the open compartments. In some embodiments, the open compartments are thus delimited by the side walls of the container and the one or more baffles.

In some embodiments, the bioreactor further comprises means for forcing, when in operation, a fluid to flow downwardly towards the at least one underflow aperture or upwardly towards the at least one overflow aperture through a preferential path.

A preferential path may be a preferential direction and orientation induced by means present along or across the flow.

A preferential flow path may be characterized by laminar flow, turbulent flow or by a combination of the two.

For example means for forcing the fluid may be means for supporting biofilms such as biofilm supports, biofilm carriers or other means for immobilizing biofilms on a substrate which may influence the flow of a fluid.

For example, means for forcing the fluid may be tubular biofilm immobilization carriers or tubular channels defining a path, e.g. inside the carrier or channel, which is preferred to another path, e.g. outside the carrier or channel.

In some embodiments, the means for forcing a fluid to flow are the biofilm carriers according to the first aspect of the invention.

In this embodiment, the preferential path is thus the one inside the hollow biofilm carrier. A preferential path does not exclude that fluid flows through other paths. However when the biofilm carriers are present the flow substantially flow throughout the carriers, i.e. more than 80% such as 85%, 90%, 95% or 99% of the total flow of the fluid flowing along the preferential path through the carriers,

Eventually paths which are not preferential clog thus leading to a 100% flow through the preferential path. On the contrary because of the configuration and position of the biofilm carriers according to the second aspect of the invention, the preferential vertical path along and inside the biofilm carriers do not clog as the fluid flow is substantially parallel to the biofilm location, formation or immobilization.

In some embodiments, the bioreactor further comprises means for promoting, such as continuously promoting, removal of precipitate, such as biomass, deposited or located on the internal surface of the bottom wall of the container.

The means for promoting removal of precipitate may be one or more rotating means, such as one or more rotating scrapers.

In another aspect the invention relates to a one or more rotating means, such as one or more rotating scrapers suitable for being used in a CSTR.

A rotating scraper may have a scraping edge and a top edge opposite to the scraping edge.

Thus in some embodiments each of the one or more rotating scrapers has a scraping edge and a top edge opposite to the scraping edge.

When not in motion, the rotating scrapers lay in a position that reduces or avoids short circuiting flow between neighbouring sections.

Thus in some embodiments, when not in motion, the one or more rotating scrapers lay in a position that reduces or avoids short circuiting flow between neighbouring sections.

For example, when not in motion each rotating scrapers may be located underneath the full baffle delimiting a section.

Thus, is some embodiments wherein when not in motion each of the one or more rotating scrapers rotating scrapers is located underneath a full baffle delimiting a section.

An opportune gap between the edge of each rotating scraper and the edge of the baffle ensures for correct rotation as well as for reducing and/or avoiding cross-flow between sections.

Thus is some embodiments there is a gap between the top edge of each of the one or more rotating scrapers and a edge of the full baffle, thereby ensuring for correct rotation as well as for reducing and/or avoiding cross-flow between sections.

During rotation, a rotating scraper rotates clockwise or counter clockwise from its resting position, e.g. underneath a full baffle, to a second resting position, e.g. underneath a second full baffle. Thus, one rotation provides scraping of the internal surface of the bottom wall of an entire section.

In some embodiments the one or more rotating scrapers are adapted to rotate clockwise or counter clockwise from a resting position underneath a first full baffle, to a second resting position underneath a second full baffle, thereby, one rotation provides scraping of the internal surface of said bottom wall of an entire section.

In some other embodiments, rotating scrapers may be located underneath low baffles and or high baffles, for example underneath each low and high baffle.

Thus in some embodiments the one or more rotating scrapers are located underneath low baffles or high baffles.

In some embodiments, the rotating scrapers provide a fluid tight seal between the bottom edge of the full baffles and the top edge of the rotating scrapers. Thus in some embodiments the one or more rotating scrapers provide a fluid tight seal between a bottom edge of full baffles and the top edge of said one or more rotating scrapers.

Fluid tight is herein defined as a seal that avoids or reduces at least by 50%, such as between 45% and 0.1%, such as between 40% and 1%, such as between 35% and 5%, such as between 30% and 10%, for example 25% the lateral flow between quarter sections of the reactor. A fluid tight seal is thus a seal that ensures that cross-flow between compartments or sections and the static zones is lower than the desired value.

A desired value for optimal operation of the bioreactor may be lower than 50, such as between 50 and 40, or lower than 30, such as between 30 and 20, or lower than 20, such as between 20 and 10, or lower than 10 such as 7, 5, 1, such as between 1 and 0.1% of the flow through the correspondent overflow and underflow aperture.

In particular, the fluid tight seal ensures that the cross-flow as indicated above is reduced to less than 10% of the flow through the correspondent overflow and underflow aperture having the effect of achieving high gas production and low retention time.

In some embodiments, the container comprises a bottom chamber defined or located between the internal surface of the bottom wall and a lowest level or lowest part of the insert according to the second aspect of the invention.

The bottom chamber may comprise the means for promoting removal of precipitate according to other embodiments of the invention.

In some embodiments, the means for promoting removal of precipitate are adapted to define, when not in operation, static zones within the bottom chamber wherein cross-flow between compartments or sections and the static zones is lower than a desired value. When in operation, the static zones become mixing zones wherein cross-flow between compartments or sections and the static zones is higher than the desired value.

A desired value for optimal operation of the bioreactor may be lower than 50, such as between 50 and 40, or lower than 30, such as between 30 and 20, or lower than 20, such as between 20 and 10, or lower than 10 such as 7, 5, 1, such as between 1 and 0.1% of the flow through the correspondent overflow and underflow aperture.

In particular, the means for promoting removal of precipitate may be adapted to reduce the cross-flow as indicated above to less than 10% of the flow through the correspondent overflow and underflow aperture having the effect of achieving high gas production and low retention time.

In some embodiments the width, or size or diameter of the insert is substantially equal to, e. g. between 0 and 5% smaller than, a width or size or diameter of the container.

The insert has to fit inside the container. Different sizes, widths and diameters are possible so as to comply with this requirement depending on the design of the bioreactor.

In some embodiments the lowest level or part of the insert is located at a desired distance from the internal surface of the bottom wall.

The desired distance is the distance allowing for reducing or avoiding short circuiting flow between neighbouring sections. The desired distance may be defined by the height of the rotating scraper, eventually allowing for a gap between the lowest level of the insert and the top edge of the rotating scraper.

In some embodiments, the bioreactor further comprises means for keeping the insert at the desired distance from the internal surface of the bottom wall.

For example, the means for keeping the insert at the desired distance may be a plurality of protrusions located on the one or more side walls of the container.

In some embodiments, the means for keeping the insert at the desired distance from the internal surface of the bottom wall are a curvature of the bottom wall. The curvature may gradually reduce the width, size or diameter of the container, wherein the width, size or diameter is defined by the one or more side walls of the container.

The insert may thus be held, raised or standing on the curvature of the bottom wall of the reactor.

In some other embodiments, where the bioreactor does not have a bottom wall that is curved other means for keeping the insert at the desired distance from the internal surface of the bottom wall may be used according to the design of the bioreactor.

In some embodiments, the open edges are displaced in respect to each other defining a plurality of underflow and overflow apertures, whereby, when the bioreactor is in operation, the fluid flows from an underflow aperture of a first compartment upwardly towards an overflow aperture of a second subsequent compartment and downwardly towards an underflow aperture of a third subsequent compartment.

In some other embodiments, the means for forcing a fluid to flow, when the bioreactor is in operation, define a preferential flow path upwardly towards an overflow aperture or downwardly towards an underflow aperture of subsequent compartments.

In some further embodiments, when the bioreactor is in operation, a cross-flow in between not subsequent compartments is lower than a desired value.

A desired value may be lower than 50, such as between 50 and 40, or lower than 30, such as between 30 and 20, or lower than 20, such as between 20 and 10, or lower than 10 such as 7, 5, 1, such as between 1 and 0.1% of the flow through the correspondent overflow and underflow aperture.

Subsequent compartments may also be neighbouring compartments that do not have favourite flow through overflow and underflow aperture in between each other. In that latter case a desire value may be lower than 50, such as between 50 and 40, or lower than 30, such as between 30 and 20, or lower than 20, such as between 20 and 10, or lower than 10 such as 7, 5, 1, such as between 1 and 0.1% of the flow through the overflow and underflow aperture of subsequent compartments having overflow and undertow aperture in between each other.

In some other embodiments, the bioreactor further comprises means for recirculating a fluid within each of the at least two open compartments.

In some embodiments, the bioreactor further comprises means for recirculating a fluid in between the at least two open compartments.

In some embodiments, the bioreactor further comprises means for recirculating a fluid within each sections.

In some further embodiments, the bioreactor further comprises means for recirculating a fluid in between sections.

In some embodiments, the means for recirculating a fluid are one or more recirculation pumps.

In some embodiments, the one or more recirculation pumps are in an amount equal to the amount of sections of the insert.

In some embodiments, the one or more recirculation pumps are in an amount at least equal to the amount of sections of the insert.

In some embodiments, the one or more recirculation pumps are in an amount equal to the amount of compartments of the insert divided by two.

The number of recirculation pumps may be more than the number of compartments. The bioreactor may be designed so as to be flexible in respect to the change of the recirculation pattern and thus of the configuration of the pumps.

In some embodiments one single pump may be used for recirculation of two or more compartments, therefore in some embodiments the pumps may be also less than the amount of compartments.

In some embodiments, the container is a cylindrical tank reactor.

The container may also have different geometries, for example the reactor may have a parallelepipedal, cubic or spherical geometry.

The invention relates in a forth aspect to a method of operating a bioreactor, the bioreactor according to third aspect of the invention, the method comprising:
feeding the bioreactor with a fluid containing biofilm precursors;
conducting digestion of the fluid.

The digestions may be aerobic or anaerobic.

In some embodiments, the fluid containing biofilm precursors is a feedstock having a COD at least 30.0 gr/L.

In some embodiments, the feedstock may have a COD higher than 30.0 gr/L.

In some further embodiments, the feedstock may have a COD lower than 30.0 gr/L.

The feedstocks may also have lower COD concentration than 30.0 gr/L. Importantly what makes the biofilm develop, is the initial growth on the carriers conducted by microbes from the Inoculum i.e. the effluent that is put inside the reactor at the start up, the gradual introduction of feedstock and the circulation flow. The biofilm develops from the bacteria present in the inoculum and also from those in the feedstock, which can be of lower and higher COD than here stated. For example, the feedstock may be waste water, e.g. having 0.5-10 g COD/L, or manures having 1-100 g/COD/L.

The feedstock may also be, for example, waste water with distillery vinasse, liquefied organic components of municipal solid waste (MSW), and wastes from abattoirs, restaurants, dairy processing, and tanneries. These waste streams contain a high level of total solids, typically greater than 7% by weight. In essence, any feedstock suitable for aerobic or anaerobic digestion/fermentation is believed to be suitable to be processed in a bioreactor as disclosed herein.

In some embodiments, the conducting digestion of the fluid occurs with a hydraulic retention time of 120 hours or less while maintaining a flow velocity of at least 0.0002 m/s and a gas production rate of at least 5.0 Lgas/Ldigester/day (litersgas/liter digester volume/day) in such a manner as to maintain a preferential flow, such as substantial laminar flow, through the biofilm carriers.

Hydraulic retention time may be between a period of 91 hours and a period of 52 days as shown in the examples. For example may be hydraulic retention time between 160 and 72 hours would also be possible as shown in example 7.

Gas production rate may be between 5.0 Lgas/Ldigester/day and 20.0 Lgas/Ldigester/day, such as between 5.0 Lgas/Ldigester/day and 15.0 Lgas/Ldigester/day, such as higher than 7.0 Lgas/Ldigester/day.

The flow velocity of at least 0.0002 m/s may be vertical, i. e. the desired flow velocity refers to the velocity of the flow in the vertical direction. Limited or absence of cross sectional flow or horizontal flow is desirable. The vertical flow is the flow along the longitudinal axes of the biofilm carrier, being the biofilm carrier located vertically along the longitudinal axis of the bioreactor.

Thus in some embodiments, the conducting digestion of the fluid occurs with a hydraulic retention time of 120 hours or less while maintaining a vertical flow velocity of at least 0.0002 m/s and a gas production rate of at least 5.0 Lgas/Ldigester/day (litersgas/liter digester volume/day) in such a manner as to maintain a substantial laminar vertical flow through the biofilm carriers.

Substantial laminar flow is defined as a flow that is mostly laminar, i.e. more than 80% such as more than 90% laminar. However, where the gas produced by the biofilm flows along the carrier walls, the substantial laminar flow may be locally turbulent, e.g. having Reynold's number between 1 and 2500.

Due to the microbial immobilisation, the system can be operated at HRT's lower than 120 hours when accepting loss in methane production efficiency. This can be the case, if treatment capacity is more important than methane yield or if an available carbon source is wanted in the effluent.

However, the system can also be operated at a retention times higher than 120 hours.

In some embodiments, the feedstock is digested under anaerobic conditions to produce biomethane with a hydraulic retention time of 120 hours, or less, such as less than 110 hours, such as less than 100 hours, less than 90 hours, less than 80 hours, less than 75 hours, less than 60 hours, less than 50 hours, or less than 40 hours or wherein the retention time is 20-120 hours, 30-120 hours, 40-120 hours, 50-120 hours, 75-120 hours, 100-120 hours, or such as 50-110 hours, 50-100 hours, 50-75 hours. while maintaining a flow velocity of at least 0.0002 m/s through the bioreactor and/or a gas production rate of at least 5.0 liters/liter digester volume/day in such a manner as to maintain a substantial laminar flow through said biofilm carriers.

In some further embodiments, the feedstock is digested under anaerobic conditions to produce biomethane with a hydraulic retention time of 120 hours, or less, while maintaining a flow velocity of at least 0.0002 m/s, such as a flow velocity between 0.0002 m/s to 0.08 m/s, such as between 0.0030 and 0.07, such as between 0.009 and 0.05, such as between 0.015 m/s to 0.045 m/s through the bioreactor and/or a gas production rate of at least 5.0 liters/liter digester volume/day in such a manner as to maintain a substantial laminar flow through said biofilm carriers.

In some further embodiments the feedstock is digested under anaerobic conditions to produce biomethane with a hydraulic retention time of 120 hours, or less, while maintaining a flow velocity of at least 0.0002 m/s through the bioreactor and/or a gas production rate of at least 5.0 liters/liter digester volume/day (L/L/D), such as in between 6.0 L/L/D and 10.0 L/L/D, such as in between 7.0 L/L/D and 9.0 L/L/D, such as at least 8.0 L/L/D.

In some embodiments, the step of conducting digestion comprises forcing the fluid to flow between the at least two compartments downwardly towards the at least one underflow aperture or upwardly towards the at least overflow aperture.

In some further embodiments, the forcing the fluid to flow further comprises forcing the fluid to flow through a preferential flow path defined by the plurality of biofilm carriers.

In some embodiments, the step of forcing the fluid to flow further comprises recirculating the fluid within each compartments.

In some further embodiments, the step of forcing the fluid to flow further comprises recirculating the fluid in between compartments.

In some further embodiments, the step of forcing the fluid to flow further comprises recirculating the fluid within each sections.

In some further embodiments, the step of forcing the fluid to flow further comprises recirculating the fluid in between sections.

In some embodiments, the method according to the fourth aspect further comprises:
removing precipitate located on the internal surface of the bottom wall of the bioreactor.

In a fifth aspect, the invention relates to a system for producing biogas, the system comprising:
at least one feed tank for feeding bioreactors;
one or more interconnected bioreactors according to the second aspect of the invention;
at least one effluent tank for collecting effluents from the one or more interconnected bioreactors.

The system may further comprise a bioreactor comprising an insert according to the first aspect on the invention.

In a sixth aspect, the invention relates to a method of converting a Continuously Stirred tank Reactor (CSTR) having an internal surface into a fixed film, fixed orientation, fixed bed anaerobic digestion reactor, the method comprising installing an insert according to the third aspect of the invention within said CSTR.

The step of installing may comprise fastening the one or more baffles to one of more locations of the internal surface of the CSTR.

Fastening may occur by means of bolt and nuts. The fastening may also occur by welding.

The step of installing may comprise, firstly inserting and fitting the insert in the CSTR and secondly installing, i.e. removably attaching, the plurality of biofilm carriers.

Installation of insert and biofilm carriers may thus occur either in one step where an insert comprising biofilm carriers is installed or in two separate steps where following the insertion of the insert, biofilm carriers are installed. Insert and biofilm carriers may be removably attached, meaning that may be attached in a way so that they can be later removed for inspections or maintenance.

In other embodiments, the method further comprising growing a biofilm within the insert.

In a seventh aspect, the invention relates to a method for performing maintenance of a CSTR modified according to the method of the fifth aspect of the invention, the method comprising: temporarily interrupting a normal operation of the modified CSTR; removing at least part of the insert; and re-installing the at least part of the insert.

At least part of the insert is removably attached so that it can be easily removed after installation.

The bioreactor on which maintenance according to the method according to the seventh aspect may comprises an insert according to the first aspect.

In a further aspect, the invention relates to a method for performing maintenance of a bioreactor according to the second aspect of the invention.

In some embodiments, at least part of the insert is at least one compartment of the insert.

The at least part of the insert may be at least one section of the insert.

The at least part of the insert may be one or more biofilm carriers within the compartments of the insert.

In an eighth aspect, the invention relates to the use of a bioreactor according to the third aspect of the invention, for producing biogas, such as biomethane.

In a ninth aspect, the invention relates to the use of a bioreactor according to the third aspect of the invention for rapid determination of a biomethane potential of a feedstock.

In a tenth aspect, the invention relates to the use of a bioreactor according to the third aspect of the invention for producing a product produced by microbial organism supported on a biofilm.

The products may be chemical or biological products, such as organic acids, hydrogen gas, farmacological or fermentative products. The bioreactor is suitable for being used in production of products that can benefit from the flow path defined by the insert and/or by the biofilm carriers.

In an eleventh aspect the invention relates to a method of aerobic or anaerobic digestion of a feedstock in the bioreactor according to third aspect of the invention, the method comprising: feeding the feedstock into the compartments of the bioreactor; —digesting the feedstock by passing the feedstock through the compartments of the bioreactor with a retention time sufficient to digest the feedstock.

The compartments may contain biofilm carriers that have been pre-inoculated to obtain a biofilm with a suitable bacterial consortium.

The bacterial consortium may be a consortium of methan-producing bacteria.

The feedstock may be mixed, or mixed at least in part between each compartment.

The feedstock may be feed simultaneously at several compartments across the bioreactor in order to form a feeding gradient.

The feedstock may be partly or completely recirculated through the bioreactor.

The feedstock may be partly recirculated between the compartments of the bioreactor.

The feedstock may be digested under anaerobic conditions to produce biomethane with a hydraulic retention time of 120 hours or less while maintaining a flow velocity of at least 0.0002 m/s through the bioreactor and/or a gas production rate of at least 5.0 liters/liter digester volume/day in such a manner as to maintain a substantial laminar flow through the biofilm carriers.

The feedstock may have a chemical oxygen demand (COD) of at least 20.0 g/L, such as at least 30.0 g/L, at least 35 g/L, at least 40 g/L or at least 50 g/L or wherein the feedstock has a COD of 20-300 g/L, 30-300 g/L, 40-300 g/L, 50-300 g/L, 75-300 g/L, 100-300 g/L, such as 25-250 g/L, 30-200 g/L, 35-150 g/L, 40-150 g/L, 50-150 g/L or such as 20-125 g/L, 30-100 g/L, 30-75 g/L, 30-50 g/L, 35-75 g/L, 40-100 g/L, 50-175 g/L, 50-200 g/L

The feedstock may be digested at a temperature between 30 and 55° C., 37 and 53° C., such as between 37 and 48° C., such as between 37 and 40° C., such as between 40 and 44° C., such as between 44 and 48° C., such as between 48 and 53° C.

The feedstock may be digested at a pH between 6.6 and 8.5, such as between 6.8 and 7.4, such as between 7.0 and 7.4, such as between 7.0 and 7.2.

In some embodiments, the pH is adjusted by recirculation and/or by addition of pH adjusting agents, such as ammonia.

PH may be adjusted also by other alkaline or acidic adjusting agents and/or through the use of buffer solutions.

The retention time is less than 110 hours, such as less than 100 hours, less than 90 hours, less than 80 hours, less than 75 hours, less than 60 hours, less than 50 hours, or less than 40 hours or wherein the retention time is 20-120 hours, 30-120 hours, 40-120 hours, 50-120 hours, 75-120 hours, 100-120 hours, or such as 50-110 hours, 50-100 hours, 50-75 hours.

In some embodiments, the flow velocity is at least 0.00025 m/s, such as at least 0.0005 m/s, at least 0.00075 m/s, at least 0.001 m/s, at least 0.0025 m/s, at least 0.005 m/s, or at least 0.0075 m/s or wherein the flow velocity is 0.0002-0.015 m/s, such as 0.0002-0.0125 m/s, 0.0002-0.01 m/s, 0.0002-0.0075 m/s, 0.0002-0.005 m/s, or such as 0.00025-0.01 m/s, 0.0005-0.01 m/s, 0.00075-0.01 m/s, 0.001-0.01 m/s, 0.0025-0.01 m/s, 0.005-0.01 m/s, or 0.0075-0.01 m/s.

The gas production rate is at least 6.0 liters/liter digester volume/day, such as 7.0 liters/liter digester volume/day, at least 8.0 liters/liter digester volume/day, 9.0 liters/liter digester volume/day, at least 10.0 liters/liter digester volume/day, such as at least 12.5 liters/liter digester volume/day, at least 15 liters/liter digester volume/day or at least 20 liters/liter digester volume/day, and/or wherein the gas production rate is 5.0-20 liters/liter digester volume/day, such as 6.0-20 liters/liter digester volume/day, 7.0-20 liters/liter digester volume/day 8.0-20 liters/liter digester volume/day, 9.0 liters/liter digester volume/day, or 10-20 liters/liter digester volume/day.

The feedstock may be a biomass.

In some embodiments, the biomass is selected from the group consisting of waste, sewage, manure, and/or a cellulosic, hemicellulosic, lignocellulosic or starch containing biomass selected from wheat straw, corn stover, sugar cane bagasse, sweet sorghum bagasse, or empty fruit bunches.

The waste is selected from the group consisting of municipal solid waste (MSW), industrial waste, animal waste or plant waste.

In some embodiments, the waste contains a level of total solids greater than 7% (w/w), such as greater than 8% (w/w), greater than 9% (w/w), greater than 10% (w/w), such as 7-20% (w/w), 8-20% (w/w), 9-20% (w/w), 10-20% (w/w), or 15-20% (w/w).

The biomass have been pre-treated by hydrothermal pre-treatment, enzymatic hydrolysis and/or aerobic digestion.

The first, second and third and other aspect, embodiment or item of the present invention may each be combined with any of the other aspects, embodiments or items. These and other aspects of the invention will be apparent from and elucidated with reference to the embodiments described hereinafter.

### BRIEF DESCRIPTION OF THE FIGURES

FIG. 1A shows one example of a bioreactor suitable for practicing methods of the invention.

FIG. 1B shows the example of a bioreactor of FIG. 1A with reference signs.

FIG. 1C shows one embodiment of the insert according to one aspect of the invention.

FIG. 1D shows another embodiment of the insert according to one aspect of the invention.

FIG. 1E shows a further embodiment of the insert according to one aspect of the invention.

FIG. 2A shows a bioreactor similar to that shown in FIG. 1, but with more detailed features, thus showing a schematics of the CSTR tank fitted with the FAD insert.

FIG. 2B shows the example of a bioreactor of FIG. 2 with reference signs.

FIG. 2C shows section of an insert according to some embodiments of the invention.

FIG. 3A shows the basic fluid flow patterns achieved in one quarter section of the bioreactor when in operation thus showing flow through a quarter section of the bioreactor shown in FIGS. 2A and 2B.

FIG. 3B shows the basic fluid flow patterns achieved in one quarter section of the bioreactor when in operation where the porous tubular biofilm carrier are shown correctly, being FIG. 3A and FIG. 3B a cross section.

FIG. 4 shows a schematic illustration of fluid flow patterns or paths, i.e. a schematic top view of two FAD digester sizes showing different chamber distributions, flow patterns and circulation flow directions and placements.

FIGS. 5A and 5B shows a porous matrix providing multiple “directions” for biomass accumulation in the biofilm.

FIG. 6 and FIG. 7 show schematic illustrations of two embodiments of a laboratory scale test device.

FIG. 8 shows Ideal Retention Time Distribution (RTD) results of “well mixed” cascading CSTR's. N=order of reactors of the species.

FIG. 9. shows experimental results of Retention Time Distribution analysis RTD graph from methylene blue passing through the three consecutive FAD digesters.

FIG. 10 shows gas production (diamonds) and feed rate (circles) in liters per day over the course of 95 days biofilm build-up of the FAD system described in example 1 using the LOF feed described in Table 2.

FIG. 11 shows COD removal and HRT vs. time during FAD load-up with liquefied organic fraction of MSW.

FIG. 12 shows a Photo of biofilm carrier with biofilm attached.

FIG. 13 shows continuous operation during rapid temperature regime changes, i. e. the gas produced per liter of 53 gCOD/L feed vs time during transition between thermophilic and mesophilic temperature range.

FIG. 14 shows the Lengthy stable FAD operation at 91 hours HRT—2 L feed/day into 7.5 L digester, showing the gas produced (201) and the feed in (202).

FIG. 15 shows the stable COD conversion efficiency during lengthy operation at 91 hours HRT.

FIG. 16 shows VFA and COD content of effluent vs time (days).

FIG. 17 shows a digester VFA concentration vs. COD conversion efficiency in the same digester.

FIG. 18 shows Biogas production before and after lengthy biofilm exposure to atmospheric oxygen.

FIG. 19 shows Gas production from lignocellulosic Thin stillage fed to the FAD digester, i. e. gas production from thin stillage from 2^{nd} generation bioethanol production fed to the FAD digester.

FIG. 20 shows gas production from pig manure fed to the FAD digester.

FIG. 21 shows gas production through a shock test.

FIG. 22 compares gas production for a single feed point and for a multi feed point for gas production (series 1) and for the substrate.

### DETAIL DESCRIPTIONS OF SOME EMBODIMENTS

In some embodiments, the invention provides a method of anaerobic digestion to biomethane comprising the steps of
introducing a substrate feedstock having COD content at least 30.0 g/L into a fixed film, fixed orientation, fixed bed bioreactor system in which the immobilization matrix is characterized in comprising a plurality of vertically oriented, porous tubular carriers supporting biofilm, and in which mixing zones are provided both above the upper openings and below the lower openings of the tubular carriers, and conducting anaerobic digestion of the feedstock with a hydraulic retention time of 120 hours or less while maintaining a flow velocity of at least 0.0002 m/s and a gas production rate of at least 5.0 liters/liter digester volume/day in such manner as to maintain a substantially laminar flow through the tubular carriers as well as mixing within each of the mixing zones.

In some embodiments, the invention provides an anaerobic digestion bioreactor comprising a cylindrical tank having a plurality of internal, vertical biofilm carrier compartments defined by baffles or walls made from corrosion resistant and liquid impermeable material that are open at the top, where in each carrier compartment comprises a shortened wall or underflow aperture on one side at the bottom which serves as an opening into another carrier compartment whereby fluid flows can be directed through succeeding compartments, and wherein a plurality of the carrier compartments further comprise a shortened wall or overflow aperture at the top on a side other than that side which contains a shortened wall or underflow aperture at the bottom which serves as an opening into another carrier compartment whereby fluid flows can be directed through succeeding compartments, optionally further comprising a rotable scraper that is adapted to define sealed sections in a sedimentation zone situated beneath the lowest edge of the carrier compartments when in a closed position or to permit removal of sedimented solids when in an open position.

In some embodiments, the invention provides an insert for converting a continuously stirred tank reactor (CSTR) into a fixed film, fixed orientation, fixed bed anaerobic digestion reactor comprising—
interconnected baffles made from corrosion resistant and liquid impermeable material that define a plurality of vertical biofilm carrier compartments that are open at the top, each of which has a shortened wall or underflow aperture on one side at the bottom which serves as an opening into another carrier compartment whereby fluid flows can be directed through succeeding compartments, and most of which have a shortened wall or overflow aperture at the top on a side other than that which contains a shortened wall or underflow aperture at the bottom which serves as an opening into another carrier compartment whereby fluid flows can be directed through succeeding compartments.

In some embodiments, the invention provides a method of converting a CSTR tank into a fixed film, fixed orientation, fixed bed anaerobic digestion reactor comprising the steps of—
assembling an insert of interconnected baffles made from corrosion resistant and liquid impermeable material that define a plurality of vertical biofilm carrier compartments that are open at the top, each of which has a shortened wall on one side at the bottom which serves as an opening into another carrier compartment whereby fluid flows can be directed through succeeding compartments, and most of which have a shortened wall at the top on a side other than that which contains a shortened wall at the bottom which serves as an opening into another carrier compartment whereby fluid flows can be directed through succeeding compartments,
installing the insert within a modified or unmodified CSTR tank,
fitting the carrier compartments defined by the insert with a plurality of porous, tubular carriers either before or after installation in the CSTR tank, and
raising a productive biofilm on the carriers.

In some embodiments, the invention provides methods and laboratory scale devices for rapid determination of biomethane potential of tested substrates.

By maintaining very high biogas production rates in fixed film, fixed orientation, fixed bed anaerobic digestion systems, biofilms can be maintained in excellent productive condition without excess accumulation of biomass and associated clogging problems. Typically biogas flows should be maintained at least at 5.0 liters total gas/liter digester volume/day (L/L/D), or at least 6.0 L/L digester volume/day, or at least 7.0, or at least 8.0, or at least 9.0. In order to achieve such high gas production, a processed waste stream typically has high COD content, at least 30.0 g/L, or at least 40.0, or at least 50.0. The range of COD content in the feed stream is typically between 20.0 g/L and 300 g/L. Total gas in this context refers to the mixed product gas comprising both carbon dioxide and methane.

COD content is determined by the ferrous ammonium sulphate method well known in the art and is expressed in mg/L or g/L.

High COD/high solids waste streams typically are associated with high content of undissolved solids. A suitable bioreactor should typically be adapted to handle undissolved solids of at least 3.0 g/L, or 5.0, or 7.0, or 8.0, or 10.0, or 15.0, or 20.0, or 25.0, or 30.0, or 35.0, or 40.0, or 45.0, or 50.0, or 55.0, or 60.0.

One approach to handling anaerobic digestion of feed stream having a high content of undissolved solids in fixed film, fixed orientation, fixed bed systems is through the use of vertically oriented immobilization matrix. Undissolved solids in a vertically oriented matrix simply precipitate along the flow path. In some embodiments, sedimenting particles can be directed into sedimentation zones where particles can be collected harmlessly.

In some embodiments, the invention provides fixed film, fixed orientation, fixed bed anaerobic digestion bioreactors comprising multiple compartments suitable for containing biofilm carrier matrix, each of which or most of which compartments is associated with a sedimentation zone. “Sedimentation zone” refers to a free volume situated between the bottom of the bioreactor tank and the lowermost edge of the carrier compartments, which are typically set significantly above the bottom of the tank. In operation, tubular biofilm carriers are typically set within the carrier compartments such that the lower openings of the carriers are situated significantly above the lowermost edge of the carrier compartments. The lowermost edge of the carrier compartments, in turn, are typically set within the bioreactor tank significantly above the physical bottom of the tank—typically between 15 and 500 cm, or between 50 and 1000 cm, depending on the size of the digester.

In some embodiments, a bioreactor of the invention is equipped with a digester bottom scraper device adapted to transport sediment formed in sedimentation zones at the bottom of the active digester volume into a sludge pump system. Sediments recovered from sedimentation zones can, in this manner, be re-introduced into the digester feed stream. This serves to extend the exposure of undissolved solids to active biomethane-producing microbiology by separating the actual retention time of undissolved solids from the overall hydraulic retention time of the feed input. In other words, undissolved solids that are precipitated from the feed stream can be recirculated without extending an otherwise short hydraulic retention time. This generally improves gas production and is in marked contrast with standard CSTR systems, in which hydraulic retention time applies to the entire feed stream, including dissolved and suspended solids.

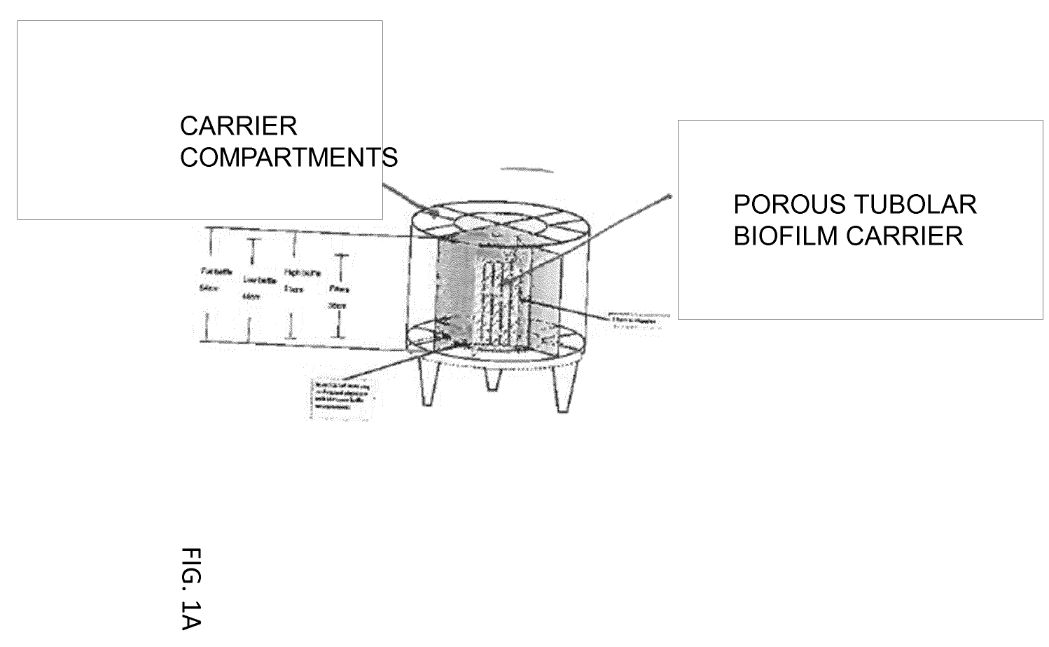

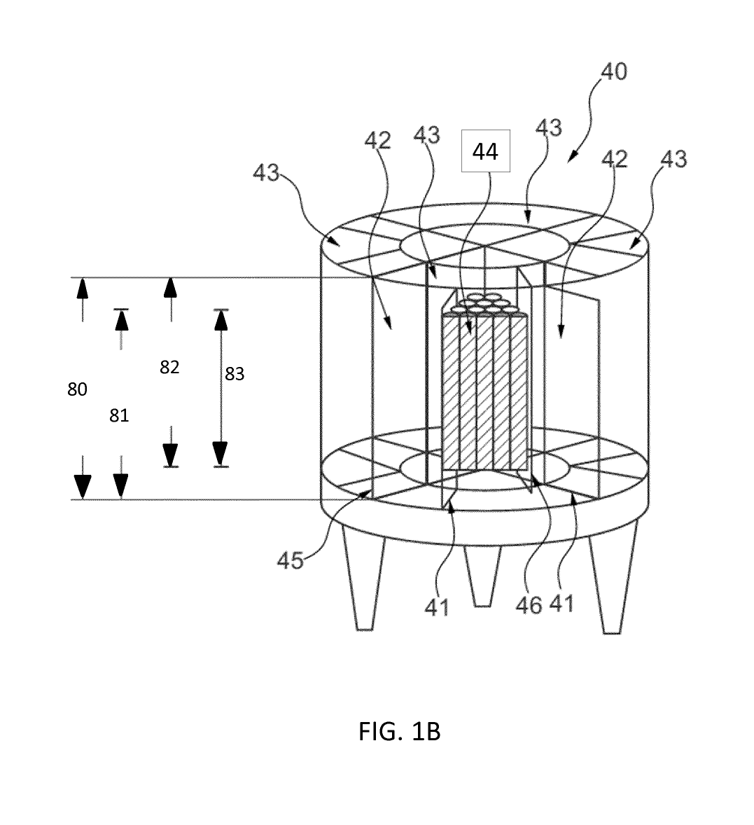

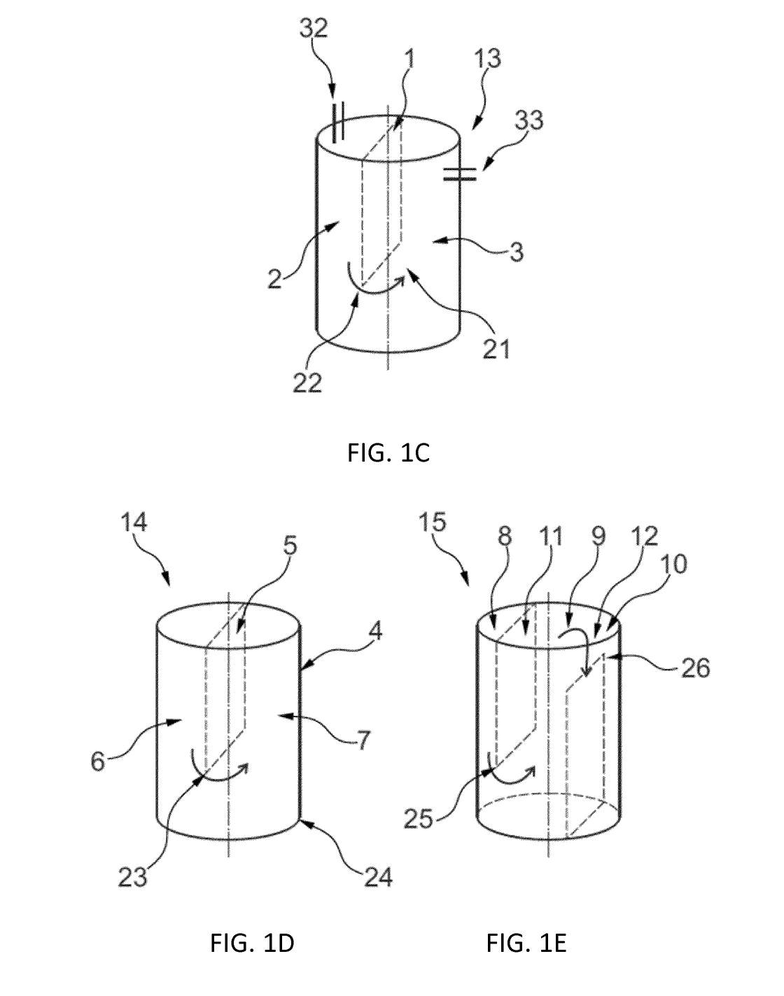

FIG. 1A shows one example of a bioreactor suitable for practicing methods of the invention. The reactor is a 300 liter CSTR tank that has been retrofitted with a system of interconnected baffles that define internal biofilm carrier compartments. As shown, the tank is divided into compartments of equal height and approximately equal cross sectional area. This ensures uniform fluid flows through the reactor. In this example, a cylindrical tank is fitted with internal compartments formed by corrosion resistant and liquid impermeable material. An inner section comprises 4 quarter-cylinder compartments having substantially equivalent cross sectional area. An outer section comprises a curved outer wall that defines a cylindrical volume and trapezoidal compartments having one curved surface formed by the outer surface of a quarter-cylinder compartment from the inner section and having one curved surface formed by the curved outer wall of the outer section. As shown, the compartments serve to contain porous, tubular biofilm carriers. In operation, the carriers are set at a level that is beneath the top wall and above the bottom wall of the compartments.

FIG. 1B shows the bioreactor 40 that is the bioreactor of FIG. 1A. The bioreactor 40 has been retrofitted with a system of interconnected baffles 41, 45, 46 that defines internal biofilm carrier compartments 42. As shown, the tank is divided into sections 43 of equal height and approximately equal cross sectional area.

All the four sections accommodate baffles of similar size.

In this example, a full baffle 45 has a height 80 of 54 cm, a low baffle 41, i.e. a baffle having an overflow aperture has a height 81 of 48 cm; a high baffle 46, i.e. a baffle having an underflow aperture has a height 82 of 51 cm. The compartments 42 accommodate porous tubular biofilm carrier 44.

The porous tubular biofilm carrier 44 has a height 83 of 35 cm.

FIG. 1C shows one embodiment of the insert according to one aspect of the invention.

FIG. 1C shows an insert for modifying a tank reactor 13, the insert comprising one baffle 1 defining two open compartments 2 and 3. The baffle comprising has an open edge 21 define an underflow aperture 22. A fluid introduced through an opening 32, such as an inlet, is forced to flow downwardly towards and across the underflow aperture 22 and eventually upwardly towards an overflow aperture or a further opening 33, such as an outlet.

FIG. 1D shows another embodiment of the insert according to one aspect of the invention.

FIG. 1D shows an insert 14 comprising two compartments 6 and 7 delimited by a continuous closed side wall 4, which is curved and surrounds the baffle 5.

The baffle 5 has an open edges 23 that is displaced in respect to a height 24 of the continuous closed side wall 4. When inserted into a reactor the flow follows the path as shown in FIG. 1C.

FIG. 1E shows a further embodiment of the insert according to one aspect of the invention.

FIG. 1E shows an insert 15 comprising two baffles 11 and 12 and three open compartments 8, 9 and 10.

The open edges 25 and 26 of the two baffles 11 and 12 are displaced in respect to each other so that when a fluid is flowing through the insert it will flow through the underflow aperture defined by open edge 25 and towards and through the overflow aperture defined by open edge 26.

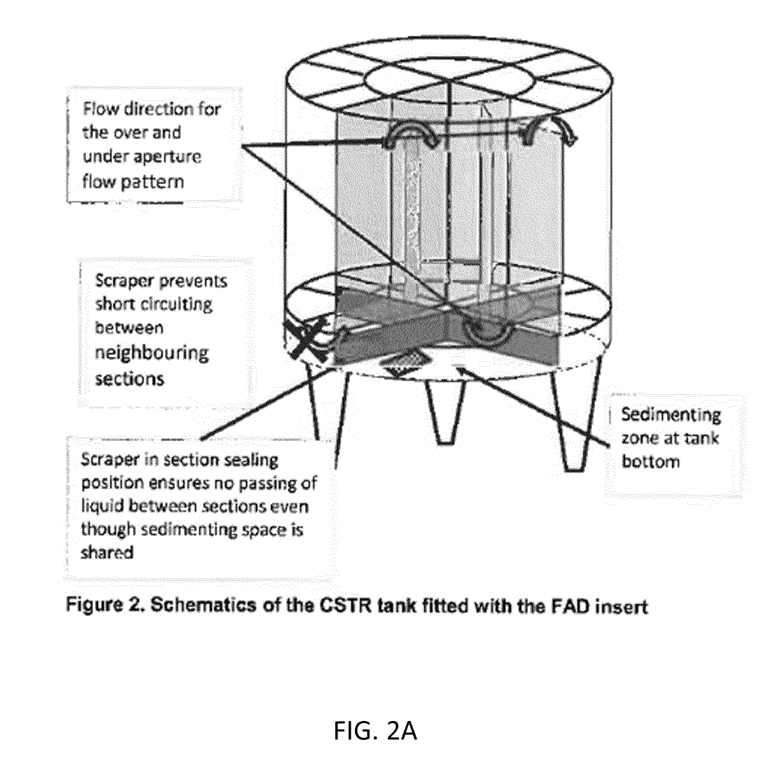

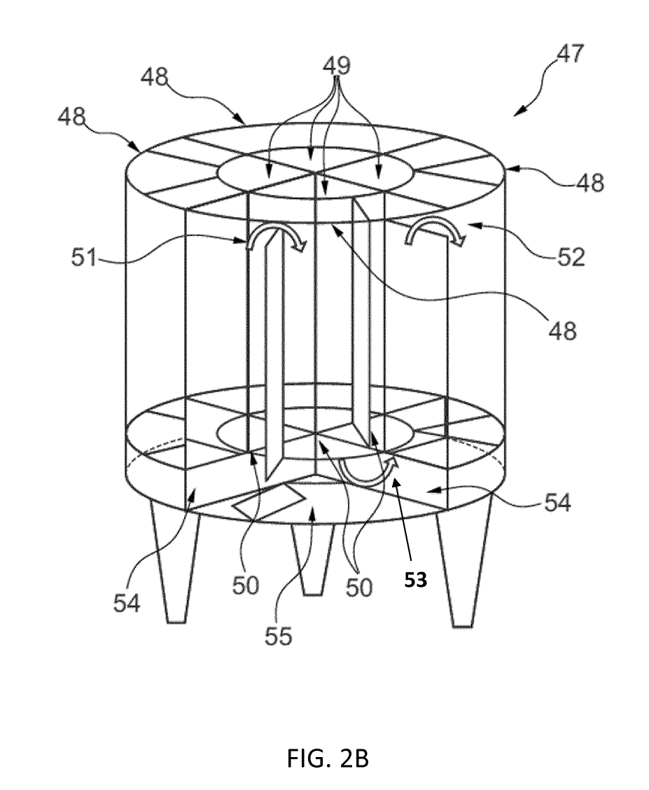

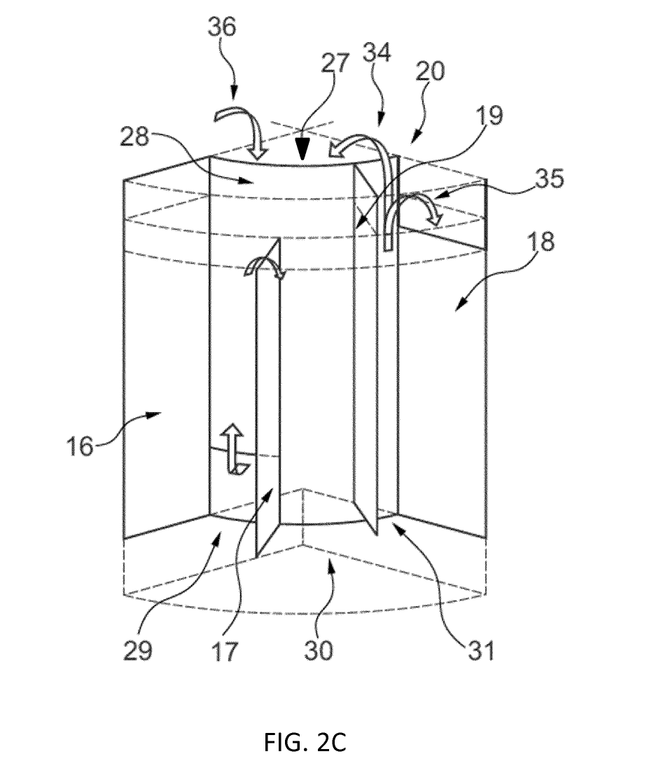

FIG. 2A shows a bioreactor similar to that shown in FIG. 1A, but with more detailed features. The digester is arranged in four quarter sections, in which each quarter-cylinder compartment of the inner section is associated with three trapezoidal compartments of the outer section. This scheme is advantageous because it is simple to assemble as an insert that can then be fitted into a larger tank, such as a CSTR tank. The compartments of the outer section become trapezoidal simply as a consequence of the geometry of the quarter-section and the division of the outer section into three compartments. In a larger scale insert for a CSTR, one or more additional outer section(s) could be included having, additional compartments.

FIG. 2B shows the bioreactor 47 that is the bioreactor of FIG. 2A. The bioreactor 47 is arranged in four quarter sections 48, in which each quarter-cylinder compartment of the inner sections 49 is associated with three trapezoidal compartments 50 of the outer section. For each outer section the fluid flow follows the directions of arrows 51, 52 and 53. The flow pattern thus is directed following the direction of over flow of arrow 51 to underflow or arrow 53 to over flow as shown by arrow 52.

Scrapers 54 prevents or reduces short circuiting between neighbouring sections.

Scraper in section sealing position ensures no passing of liquid or reduces passage of fluids between sections even though sedimentation space is shared.

Sedimentation zones 55 are located at the bottom of the bioreactor 47.

FIG. 2C shows section of an insert according to some embodiments of the invention.

FIG. 2C shows a section of an insert 20 having interconnected baffles 16, 17, 18 and 19. The baffles are a full baffle 16, two low baffles 17 and 18 and a high baffle 19.

The insert 20 has an inner or internal section 27 and an outer or external section 28. The outer section 28 comprises three open compartments 29, 20 and 31, in between baffles 16 and 18 and defined by baffles 17 and 19.

The insert 20 forces a fluid inserted, according to the direction of arrow 36, in section 27 to flow downwardly towards the underflow aperture leading to compartment 29 and then upwardly towards the overflow aperture leading to compartment 30. In compartment 30 the fluid flows downwardly towards the underflow aperture leading to compartment 31 and upwardly according to the direction of arrows 34 and 35 back into sections 27 or out of the section and insert according to the direction of arrow 35.

Fluid flow is directed sequentially through succeeding carrier compartments within each quarter section by a system of overflow and underflow apertures. Re-circulation suction pumps are provided for each quarter section. The pumps are adapted to withdraw fluid from the top of the last compartment within the flow sequence of a quarter section, in which the vertical direction of flow is upward. This removed fluid is then re-introduced to the first compartment within the flow sequence of the quarter section. The recirculation flow can be introduced from above the surface of liquid in this compartment, thereby actively enhancing mixing in the chamber to which the recirculation flow is introduced. Influent feed stream is mixed with recirculation flow. This in turn drives fluid flow through the reactor—the net volume of feed stream introduced drives net flow through the reactor.

A feed inlet introduces feed stream mixed with recirculating effluent into one quarter-cylinder compartment of the inner section. The curved wall of this compartment is shortened at the bottom, providing an opening for fluid flow into the bottom of a first trapezoidal compartment of the outer section. This shortened wall is one means for achieving an underflow aperture, meaning an opening at the bottom of the compartment that permits fluid flow into a succeeding compartment. Similarly a shortened wall at the top of a compartment is one means for achieving an overflow aperture whereby fluid flow is directed into succeeding compartments. Underflow or overflow apertures may alternatively be simply an opening in an otherwise intact wall. However, this arrangement increases the risk of channelling. In operation, tubular biofilm carriers are typically set in compartments at a level such that the lowermost openings of the tubular carriers are at a height above the underflow aperture (i.e. above the lowermost surface of the shortened wall at the bottom) corresponding to between 2-10 times the diameter of the carriers' primary fluid channel. Similarly, the uppermost openings of the tubular carriers are at a height below the overflow aperture (i.e. below the uppermost surface of the shortened wall at the top) corresponding to between 2-10 times the diameter of the carriers' primary fluid channel. This placement minimizes the risk of channelling as flows enter into or emerge out of the biofilm carriers and further defines mixing zones both above the uppermost openings and below the lowermost openings of the carriers.

The physical carrier compartments themselves are set within the bioreactor tank at a level such that the lowermost edge of the compartments is above the physical bottom of the bioreactor tank. The open volume beneath the lowermost edge of the carrier compartments defines a sedimentation zone in which sedimenting particles can accumulate. The embodiment shown in FIG. 2A is further equipped with a rotable scraper having four arms, each of which arms, in the closed position, makes a fluid tight seal between two quarter-sections of the cylindrical tank by sealing the gap between the lowermost edge of the carrier compartments and the bottom of the tank. This fluid tight seal ensures that fluids within the sedimentation zone will not flow laterally between quarter sections of the reactor. The rotable scraper can be used periodically to force sediment into a sediment outlet from which it can be pumped and recirculated into the feed stream.

Feed stream mixed with recirculated liquid enters a first compartment of the inner section, travels in a downward vertical direction through biofilm carriers, then passes through the underflow aperture into a trapezoidal compartment of the outer section. The fluid flow through biofilm carriers in the second compartment is forced into an upward vertical direction. At the top of this second compartment, the fluid flow passes through an overflow aperture into a third compartment. Here again, the fluid flow is forced to change vertical direction into a downward vertical flow through the third compartment. In this manner, the flow is forced into a pattern of alternating downward and upward direction and routed sequentially through each compartment of the reactor until it reaches the last compartment of the sequence, which is fitted with an effluent outlet that is situated at a level intermediate between the top surface of the compartments and the level corresponding to the bottom of the overflow apertures, i.e. the uppermost surfaces of the shortened walls at the top of compartments. This ensures that effluent will be driven out by force of gravity.

In some embodiments, the flow within each quarter section is continuously recirculated. The volume of feed stream introduced ensures that there will be net displacement sequentially between the quarter sections and out through the effluent outlet, notwithstanding continuous recirculation within each quarter section.

The region beneath the lowermost openings of the biofilm carriers at the bottom of two compartments which are in fluid communication via an underflow aperture provides a mixing zone. The region above the uppermost openings of the biofilm carriers at the top of two compartments which are in fluid communication via an overflow aperture similarly defines a mixing zone. In some embodiments, mixing is achieved within the mixing zones during operation by the forced change of vertical direction of flow from downward to upward. Because fluid flow through the reactor is achieved without agitation, the flow through the tubular biofilm carriers is substantially laminar. Furthermore, without agitation, undissolved particles will precipitate down the tubular biofilm carriers' primary, vertical fluid channel and into the sedimentation zones.

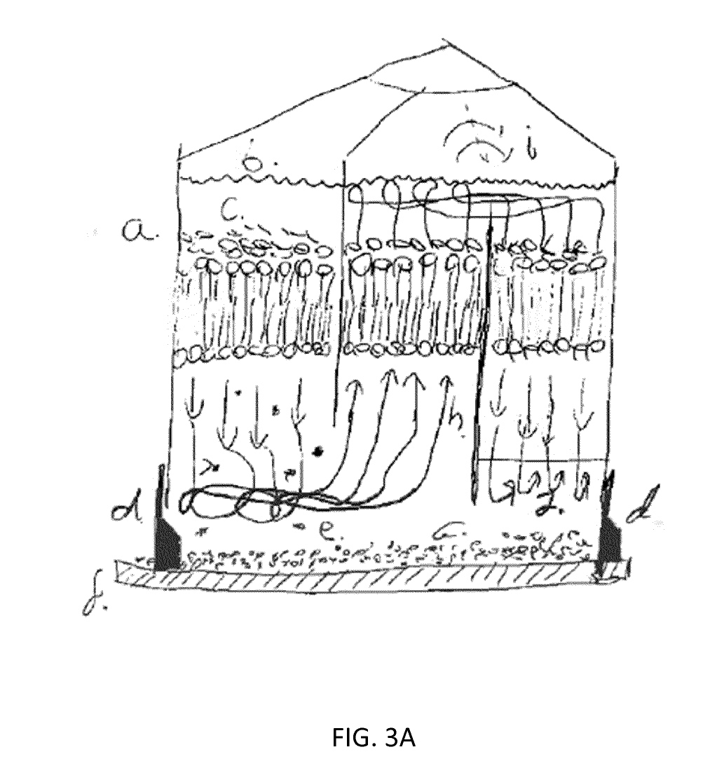

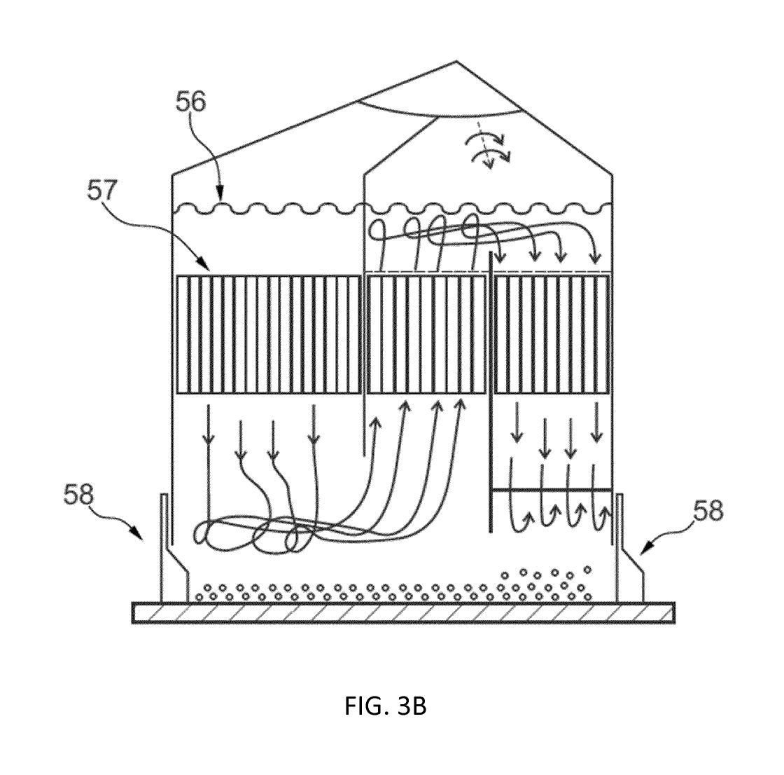

The basic fluid flow patterns achieved in one quarter section of the bioreactor shown in FIG. 2A, in operation, are illustrated in FIG. 3A. Note that the relative size of compartments and open volumes are not drawn to scale such that the illustration is only rough. As shown, a downward vertical flow is directed through a first compartment in the quarter section (b), passing through tubular biofilm carriers as a plug-flow. Sediments form in a sedimentation zone that has a shared open volume for all compartments of the section. A sludge scraper blade (d) seals the volume between the lowermost edge of the carrier compartment and the bottom of the tank, preventing fluid from flowing along the bottom between quarter-sections of the bioreactor tank. Downward flow through the first compartment is changed into an upward vertical flow through the second compartment. An underflow aperture is provided by a shortened wall at the bottom of the first and second compartments. A mixing zone is provided in the open volume beneath the lower openings of the tubular biofilm carriers. Suction action of recirculation pumps situated in the last chamber in the flow sequence of the section serve to draw flow. A gentle mixing is accomplished in the mixing region at the bottom. Upward vertical flow through the second compartment is changed into a downward vertical flow through the third compartment. An overflow aperture is provided by a shortened wall at the top of the second and third compartments. A mixing zone is provided in the open volume above the upper openings of the tubular biofilm carriers. Suction action of recirculation pumps situated in the last chamber of the section serves to draw flow. A gentle mixing is accomplished in the mixing region at the top. Downward vertical flow through the third compartment is changed into a upward vertical flow through the fourth compartment. An underflow aperture is provided by a shortened wall at the bottom of the third and fourth compartments.

FIG. 3B shown as in FIG. 3A, a downward vertical flow is directed through a first compartment in the quarter section 56, passing through tubular biofilm carriers 57 as a plug-flow. Sediments form in a sedimentation zone that has a shared open volume for all compartments of the section. A sludge scraper blade 58 seals the volume between the lowermost edge of the carrier compartment and the bottom of the tank, preventing fluid or reducing fluid from flowing along the bottom between quarter-sections of the bioreactor tank.

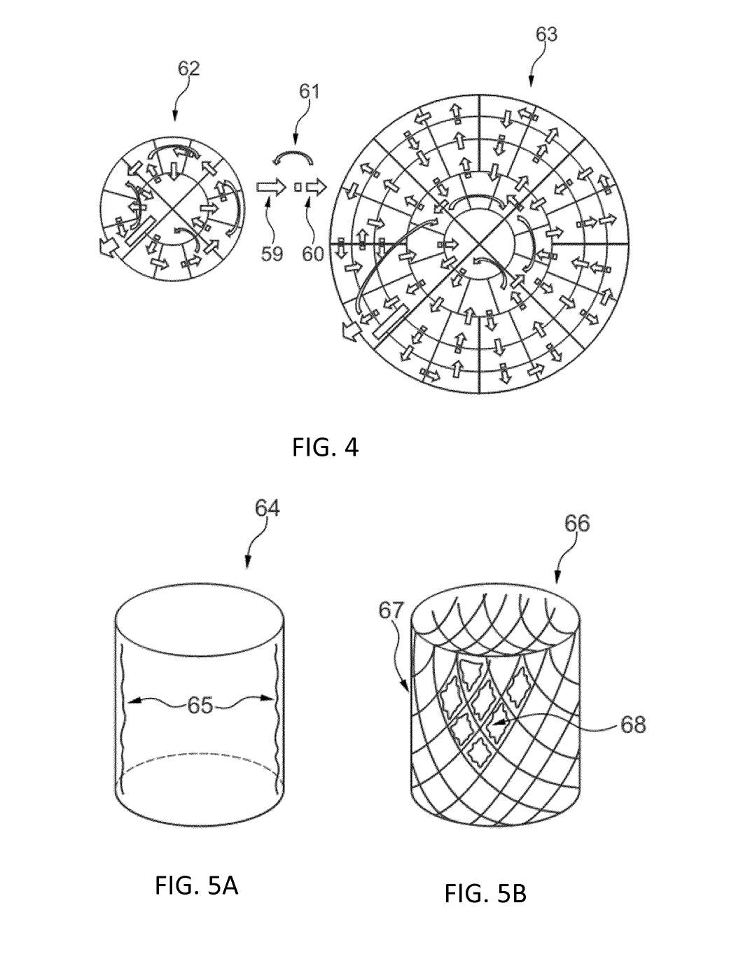

FIG. 4 shows a schematic illustration of fluid flow patterns, through alternating underflow and overflow apertures (i.e., in alternating upward and downward vertical flow) between carrier compartments of the bioreactor described in FIG. 2A and for a similar bioreactor scaled to 1000 m³ size. The top view shows the chamber distribution, the flow pattern with over- and underflow and the circulation flow direction and placement. Both examples are CSTR tanks fitted with an insert of the invention in which the carrier compartments are filled with tubular biofilm carriers.

In FIG. 4 the flow is shown as overflow according to arrow 59, as underflow according to arrow 60 and as circulation direction and placement according to arrow 61 for two different type of reactors, being 62 a FAD 0.3 m³ and 63 an FAD 1000 m³.

In embodiments such as those described in FIG. 2A and FIG. 4, in operation, liquid flow through the upper mixing zones is defined by the walls of carrier compartments being higher than the liquid surface. Liquid flow through the lower mixing zones is not limited by compartment walls or baffles. Instead, liquid flowing out of one compartment at the bottom will be sucked into the up-going flow through the next compartment. The sedimentation zone underneath the lowermost edge of the carrier compartments is one shared volume between the lowermost edge of the carrier compartments and the digester tank bottom. from the lowest chamber walls to the digester tank bottom. Liquid flow leaving the biofilm carriers in the first compartment in the flow sequence of a quarter-section will travel towards the digester tank bottom. As suction in the second compartment in the flow sequence removes liquid beneath the biofilm carriers in the second compartment, an equivalent volume of liquid will be replaced by a path of least resistance—from liquid passing through the underflow aperture between the first and second compartments. No matter whether the liquid entering the second compartment travels directly from the first compartment to the second compartment or whether it passes through some part of the sedimentation zone at the digester tank bottom, the liquid entering a biofilm carrier in the second compartment will have been mixed between the exit from the first compartment to the entrance to the second compartment. The sedimenting particles that do not follow the liquid flow through the compartment will be allowed to collect at the tank bottom until the digester bottom scraper transport them to sediment removal. Similar mixing occurs within the mixing zone at the top of the second and third compartments in the flow sequence of a quarter-section. As suction in the third compartment in the flow sequence removes liquid above the biofilm carriers in the third compartment, an equivalent volume of liquid will be replaced by a path of least resistance—from liquid passing through the overflow aperture between the second and third compartments. The pattern of alternating upward and downward vertical through succeeding compartments in a quarter-section thus achieves mixing both in the mixing zones above the biofilm carriers and also in the mixing zones below the biofilm carriers between two adjacent compartments.

In practicing methods of the invention, the carrier matrix used to support biofilm in a fixed film, fixed orientation, fixed bed system is ideally tubular and porous. The term “tubular” as used herein refers to a structure that defines one or more central channels through which fluid will flow in one direction by the force of gravity when it is placed in an upright, vertical orientation. A tubular matrix can have one or more central channels having an irregular, rectangular or even triangular cross sectional geometry. However, tubular matrix is preferably cylindrical, that is, having one or more central channels having a circular cross sectional geometry. Cylindrical geometry is preferable because the presence of corners in the fluid channel creates pockets of restricted flow. This in turn tends to promote accumulation of biomass and even sediment in the restricted flow areas, which both reduces the active surface of biofilm and also increases the risk of channelling effects.

The biofilm carrier matrix is preferably porous. The term “porous” refers to a carrier matrix having openings on the channel-forming surface which may be openings formed between twisting and evaginated surfaces. A smooth surface matrix, for example, the CLOISONYL™ tubes used by Escudie et al. (2011), permit only one possible “direction” for biomass accumulation in the biofilm—towards occlusion of the biofilm carrier's fluid channels. In contrast, as illustrated in FIG. 5B, porous matrix provides multiple “directions” for biomass accumulation in the biofilm, and tends to promote growth in a thinner film in which surface area to volume ratio of the film is maximized.

FIG. 5A shows the smooth surfaced biofilm carrier versus threaded, corrugated biofilm carrier, wherein the smooth surface biofilm carrier 64 carries a vulnerable biofilm 65 attached to the smooth surface, i.e. the biofilm 65 can be easily torn off.

The porous biofilm carrier 66 has carrier walls 67 consisting of threaded material. The biofilm 68 attaches to all surfaces of the threads. If the biofilm pointing inwards in the tube is torn, the biofilm attached to the other dimensions of the thread remains attached, thus being able to regenerate the biofilm washed away.

In some embodiments, a porous biofilm carrier matrix has a total surface area to volume ratio of between 60 m²/m³ and 300 m²/m³, or between 80 and 200, or between 90 and 150. The total surface area to volume ratio of the carrier matrix is defined by the nominal total volume of the channel-forming matrix, as defined by its outer-most boundaries, and by the exposed surface area of the matrix prior to biofilm accumulation. In some embodiments, the central channel of a porous biofilm carrier matrix as a percentage of cross-sectional area prior to biofilm accumulation is between 40% and 80%, or between 50% and 70%, or between 60% and 65%. In some embodiments, the percentage of void volume of the total volume of a porous biofilm carrier matrix is between 50% and 90%, or between 60% and 88%, or between 72% and 82%. In some embodiments, the tube diameter of a porous, cylindrical biofilm carrier matrix is between 0.030 m and 0.080 m, or between 0.036 and 0.070, or between 0.04 and 0.055.

Suitable material for use as immobilization matrix may include polyethylene, polypropylene, nylon, ceramics and most other materials that are resistant to acid and alkali corrosion and that will allow for bacterial exopolymer to attach to the carrier.

In some embodiments, a matrix comprising netting is used in which the netting is formed into a tube and in which the netting defines the outer periphery of the total volume. In some embodiments, the netting is formed by intertwined, extruded polyethylene threads having surface roughness. The roughness of carrier threads promotes microbial adherence as it presents small crevices and holes in which microbes may attach. Netting also renders biofilm resilient to dis-attachment by ordinary shear forces compared with a biofilm carrier having a smooth surface. Where the biofilm carrier is formed from rough netting, high flow velocity is less likely to increase risk of biofilm disruption, and the related risk of clogging. One suitable, commercially available material for use as biofilm carrier formed by netting are the various forms of BIO BLOK™ provided by EXPO NET™, including BIO BLOK 80™, BIO BLOK 100™, BIO BLOK 150™, BIO BLOK 200™ and BIO BLOK 300™.

Methods of the invention are practiced using a plurality of vertically oriented, porous, tubular carriers supporting biofilm. In order to develop a biofilm suitable for practicing methods of the invention, start-up and initiation procedures known in the art may be used, including but not limited to those described by Hickey et al. 1991. Cell density of microorganisms within a biofilm formed on a carrier can typically reach levels one order of magnitude higher than can be achieved in CSTR liquid volumes. See Langer et al. (2014). It is advantageous to develop a biofilm having a high relative proportion of Archaea to bacteria. This can typically be achieved by use of high VFA feeding, as described by Hickey et al. 1991, where COD from VFA is at least 20 g/L in the start-up feed stream. It is further advantageous to use a high COD organic load in the start-up feed stream, wherein total COD is at least 30 g/L, or between 35-15o g/L, and wherein organic load is taken to levels of at least 50 g/L digester volume/day. The biofilm advantageously has a relative proportion of methanogenic Archaea relative to bacteria of at least 25%, or at least 30%, or at least 31%, or at least 32%, or at least 33%, or at least 34%, or at least 35%, or at least 36%, or at least 37%, or at least 38%, or at least 39%, or at least 40%, or at least 41%, or at least 42%, or at least 43%, or at least 44%, or at least 45%, or at least 46%, or at least 47%, or at least 48%, or at least 49%, or at least 50%.

The relative proportion of Archaea to bacteria in the biofilm is determined in a biofilm sample by comparing the products from 16srRNA polymerase chain reaction (PCR) using universal 16s rRNA and Archaea-specific 16s rRNA primers reported by Gantner et al. (2011) in a DGGE gel.

In the bioreactor which contains the plurality of carriers, there are ideally “mixing zones” both above the upper openings and below the lower openings of the carriers, where “openings” refers to the central channel through which fluid flow emerges at the bottom surface of the tubular structure which defines the channel. “Mixing zone” refers to an open volume in which mixing can be achieved outside the carrier channel volume in which fluid flow should be substantially laminar and, thus, substantially unmixed, except for some back-mixing at the biofilm surface.

Flow is said to be substantially laminar where the corresponding Reynolds number is 3200 or lower. As is well known in the art, Reynolds number is a dimensionless parameter used to predict flow patterns within defined physical constraints. Reynolds number is calculated from a ratio of inertial forces to viscous forces under defined flow conditions. For example, in the specific case of fluid flow through a pipe, which is analogous to fluid flow through the central channel of a tubular biofilm carrier having cylindrical geometry, the Reynolds number is defined as Q*Dh/vA, where Q refers to volumetric flow in m³/s, Dh refers to the hydraulic diameter, meaning the effective internal diameter of the channel defined by the tubular carrier, v is the kinematic viscosity in m²/s (calculated as the ratio of the fluid viscosity in kg/m*s to its density in kg/m³), and A is the effective cross-sectional area of the internal diameter of the channel in meters (m). It is possible to calculate an upper limit to the possible Reynolds number under any given flow circumstances for anaerobic digestion feed stream by using the kinematic viscosity of water at an appropriate temperature, since this is invariably smaller than the corresponding value for high solids feed streams.

Generally the flow through tubular carriers will remain substantially laminar so long as the flow velocity through each carrier does not give rise to Reynolds number higher than 3200 Flow is said to be substantially laminar meaning that the flow pattern is expected to be laminar, however, some back mixing may occur as a consequence of biogas production or for other reasons. In order to achieve an even fluid velocity distribution and substantially laminar flow through the tubular carriers, the total carrier cross sectional area will be limited by the chamber dimensions. Flow velocities through systems of the invention are determined by inter-relationships between dimensions of the carrier compartments and capacity of circulation pumps. Bioreactors of the invention typically permit one circulation pump to circulate many compartments. As digester size and digesting capacity increase, the number of carrier compartments increases.

In some embodiments, control of flow through a bioreactor of the invention can be described as follows. To secure the correct flow in the embodiments shown in FIG. 2A and FIG. 4 through the biofilm carrier chambers and overall, through the entire digester, each of the four quarter-sections of the FAD bioreactor is circulated by a circulation pump. The circulation system thus comprises 4 equivalent liquid circulation pumps which control circulation between carrier compartments within a quarter-section. Circulation serves two purposes; securing the correct flow velocity through the biofilm carriers and re-introducing biomass that has just passed through one carrier compartment into the next compartment in sequence at an adequate frequency. The greater is the total number of carrier compartments in the bioreactor, the higher will be the flow velocity in the biofilm carriers at any given circulation pump flow. The principle is that each time the digester volume is split into two equal volumes by means of a vertical fluid barrier, the flow velocity in each half of the digester volume will be doubled relative to the flow velocity in the undivided digester volume, provided that the flow is forced to travel along the vertical height of both halves. When the digester volume is equally divided into X sections, the circulation pump flow in each section is then X times faster than the overall circulation flow through the digester. This enables both the design of a specific flow velocity through each chamber and limits the necessary circulation pump capacity.

The capacity of the circulation pumps should ideally be enough to re-introduce the circulation flow into each of the carrier compartments within a quarter-section at least two times per hour. In some embodiments, capacity of recirculation pumps is sufficient to re-introduce circulation flow into each of the carrier compartments within a quarter-section between 2 and 30 times per hour, or between 3 and 20. The required flow velocity and the minimum volume re-introduction requirement define the maximum and minimum circulation pump capacity for any given size of FAD digester. As the feed flow is introduced into one or more of the four quarter-section circulation streams, the feed flow contributes to the overall biofilm carrier flow velocity and should generally be taken into consideration when determining the correct circulation pump flow capacity.

TABLE 1
Chamber and circulation pump design parameters for FAD digesters

| | | | Fad | Fad |
|---|---|---|---|---|
| Parameter | Fad1 | Fad2 | 100 m³ | 1000 m³ |
| Tank r (m) | 0.090 | 0.370 | 2.000 | 5.200 |
| Height (m) | 0.40 | 0.54 | 8.00 | 12.00 |
| Circulation pump | 27.00 | 300.00 | 12000.00 | 20000.00 |
| flow (L/h) | | | | |
| Feed flow (L/h) | 0.31 | 2.10 | 1380.00 | 13800.00 |
| No. of Chambers | 1.00 | 16.00 | 16.00 | 64.00 |
| Tank cross area (m²) | 0.025 | 0.43 | 12.56 | 84.91 |
| Tank Vol. (m³) | 0.010 | 0.23 | 100.48 | 1018.87 |
| Chamber cross area (m²) | 0.025 | 0.027 | 0.79 | 1.33 |
| Chamber Vol. (m³) | 0.010 | 0.015 | 6.280 | 15.920 |
| Tank vol. exchange (h) | 0.4 | 0.8 | 7.5 | 30.1 |
| Section volume | N/A | 0.2 | 1.9 | 7.5 |
| exchange (h) | | | | |
| Chamber reintroduction | 2.7 | 20.8 | 2.1 | 2.1 |
| (1/h) | | | | |
| Tank flow velocity (m/s) | 2.98E−04 | 1.95E−04 | 2.96E−04 | 1.11E−04 |
| Chamber flow velocity | 2.98E−04 | 3.12E−03 | 4.73E−03 | 7.08E−03 |
| (m/s) | | | | |
| Reynolds number in | 7 | 137 | 208 | 311 |
| biofilm carrier tubes | | | | |

Any size and type of CSTR tank can be fitted with an insert to make a bioreactor of the invention. The dimensions, circulation flows and chamber arrangement will differ and can be adapted to each tank type as described in Table 1. It will be readily understood by one skilled in the art that other schemes for compartmentalization may be used in addition to the quarter-section scheme of embodiments shown in FIG. 2A and FIG. 4.

It is advantageous to achieve mixing of fluids both before and after they pass through the biofilm carriers. Flow through the central channel of the carriers is typically a plug-flow. This flow will only experience a slight back-mixing within each separate tubular carrier as the plug flow progresses. Each tube will then experience a flow front that is characterized by having a Gaussian distribution of different velocities, with that portion of the liquid flow through the center part of the channel having a higher velocity than that portion of the liquid flow that is in close proximity with the carrier “walls,” i.e. with the biofilm surface.

When the flow through each tubular carrier exits the carrier, the periphery of the flow near the biofilm surface will have had a longer dwell time in the tube than that part of the flow in the center of the channel. The periphery flow will thus have had a much better chance of exchanging substrate and products with the biofilm than will the flow in the center of the channel. In order to avoid a situation where the same central flow that exits one carrier enters a subsequent carrier in a compartmentalized bioreactor again in the center of the carrier channel, the flowing fluid should ideally be mixed when passing from one compartment to another. When using an anaerobic digestion reactor of the invention, the direction of vertical fluid flow through biofilm carriers alternates between succeeding biofilm carrier compartments between “down” and “up.” The liquid passing through the biofilm carrier in an up/down direction will be transferred to the next compartment via a horizontal movement. Thus when down flow enters a sedimenting zone below the lower openings of the biofilm carriers, the flow will be forced sideways through the sedimentation zone to the volume under the succeeding biofilm carrier chamber. The sideways movement of the flow, which—until this point has been vertical—achieves a gentle mixing of the liquid prior to its being forced upwards in a plug-flow through a biofilm carrier in a succeeding biofilm carrier chamber.

Mixing is achieved in mixing zones, and can be accomplished by a variety of different means. In some embodiments, sedimentation zones are themselves mixing zones. In some embodiments, mixing may be achieved by a mixing pump or an agitator. In some embodiments, mixing in some compartments of a bioreactor may be achieved by introducing a feed stream and/or recirculation stream from above the fluid surface, thereby achieving a splashing mixing effect. In some embodiments, mixing is achieved simply by forcing the fluid flow into a volume from which it is forced to change its direction of vertical flow.

Anaerobic digestion is conducted by means well known in the art, but informed by new results presented here. We have discovered that, using fixed film, fixed orientation, fixed bed systems in which the biofilm was developed using high VFA feed, the anaerobic biofilm is resistant to all manner of phenomena that are normally toxic in CSTR systems, including high salt content, high VFA content, and oxygen exposure. Further, and surprisingly, the operation temperature is in fact readily changeable, notwithstanding prevailing prior belief that anaerobic digestion microbes cannot simply be rapidly shifted from mesophilic (35-42° C.) to thermophilic (49-55° C.) conditions. See e.g. Bouskova et al. (2005) and see Li et al. (2014). Our results demonstrate that in fixed film, fixed orientation, fixed bed systems, such a rapid shift is in fact readily possible.

In order to achieve very high total gas production rates, the high solids feed stream is typically processed within a short hydraulic retention time (HRT), 120 hours or less, or 100 hours or less, or 75 hours or less.

Further, to maintain high total gas production, an appropriately fast flow velocity is maintained. “Flow velocity” as used herein refers to the linear velocity of fluid flow through the tubular biofilm carriers, expressed in meters/second (m/s). Flow velocity can be controlled by a variety of means, as will be readily understood by one skilled in the art. In some embodiments, flow velocity is controlled by the total influent input including both feed stream and recirculation. For example, when methods of the invention are practiced using a reactor that is compartmentalized so as to comprise a plurality of equally sized biofilm carrier compartments which are filled with tubular carriers supporting biofilm, flow velocity can be approximated as follows: ( 1/3600 seconds/hour)*[total input in liters/hour (including feed stream input and recirculation stream)/total digester usable internal volume in liters (which is defined by the total volume of liquid in the digester tank minus the net volume displacement of liquid by the tubular carriers)]*(height of the liquid column in the digester in m)*(total number of biofilm carrier compartments in the digester).

In some embodiments, for example, when using a small sized reactor as a laboratory scale device for determination of biogas potentials of tested substrates, flow velocity should be maintained at least at 0.0002 m/s or higher. In a 1000 m³ commercial scale reactor, flow velocity should be maintained at much higher rates at least 0.020 m/s. Typically, flow velocity should be maintained within the range 0.0002 m/s to 0.08 m/s, or between 0.0030 and 0.07, or between 0.009 and 0.05.

Other embodiments of a bioreactor of the invention may have other shapes of digester chambers. One such alternative chamber shape could be rectangular shaped chambers, round chambers, hexagonal or octagonal chambers. The chambers can take on any shape that both allow for the chambers to occupy the whole of the digester cross section area and prevent sharp flow-slowing corners.

Sediment typically has between 12-15% by weight dry matter, where “dry matter” refers to total solids, and typically comprises a substantial component of biologically inert, i.e. undigestible, COD and inorganic dry matter, primarily inorganics that were freed from the feed stream biomass during digestion. Sediment obtained from such systems typically offers good fertilising power in that it contains most of the phosphorous content from the feed stream as well as a high concentration of nitrogen-containing compounds and nutrient salts. When dewatered further, for example, by means of decanting, filtration, evaporation or other means known in the art, sediment obtained from such systems can have between 30-50% dry matter, which reduces handling costs when the material is discarded, transported for use as fertiliser or incinerated.

A smaller, simpler version of a reactor suitable for practicing methods of the invention can be used as a laboratory scale device for rapid determination of biomethane potential of tested substrates. It is generally accepted by those skilled in the art that biomethane potentials determined in 20-week long laboratory batch tests inevitably overestimate the yields that can actually be achieved in a commercial scale CSTR system. Typically these laboratory figures are deflated by 20% in calculation of commercial expectations. In contrast with batch CSTR tests, however, the fixed film, fixed orientation, fixed bed systems of the invention provide biomethane potential estimates on laboratory scale that very nearly approximate the yields that can be achieved using these systems in commercial scale. Moreover, unlike CSTR batch tests, which are time consuming, biomethane potential tests using systems of the invention can deliver accurate measurements within a single week.

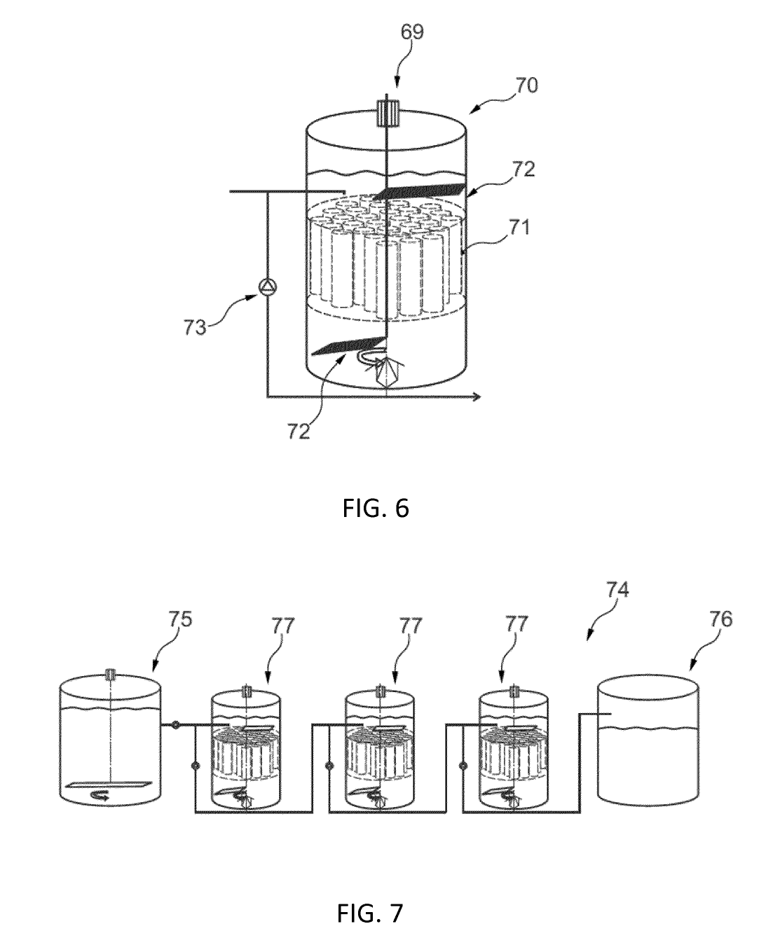

FIG. 6 and FIG. 7 show schematic illustrations of two embodiments of a laboratory scale test device. The reactor shown in FIG. 6 is a single cylindrical tank 70 with mixing zones above and below vertically oriented tubular biofilm carriers 71. Each of the mixing zones is agitated by a rotor 69 having blades 72. The liquid content of the tank are continuously recirculated according to the circulation flow 73. Influent feed is mixed with recirculated liquid. The net volume of added feed determines net displacement of effluent from the system. The reactor shown in FIG. 7 is somewhat more complex in that three individual reactors of the type shown in FIG. 6 are combined in series. Thus, FIG. 7 shows a bench-scale FAD digester in series resembling three FAD chambers. The liquid content of each of the tanks is continuously recirculated. The net volume of added feed determines net displacement from the first tank to the second and from the second tank to the third to effluent. In this arrangement, the bulk of anaerobic digestion occurs in the first tank, but good finishing is achieved in later tanks in the series. This generally mimics the circumstances of a commercial scale plant.

Thus FIG. 7 shows a system for producing biogas (74), the system comprising: at least one feed tank (75) for feeding bioreactors; one or more interconnected bioreactors (77); at least one effluent tank (76) for collecting effluents from the one or more interconnected bioreactors.

### Example 1. Design of 30 Liter Reactor System

A 30 L biogas bioreactor system termed “Fast Anaerobic Digestion (FAD)” system was designed comprising a feed tank, three consecutive anaerobic digesters and an effluent tank. Each of the three consecutive digester tanks was equipped with non-random vertically oriented tubular bacteria carriers, BIO BLOK 300™, on which an anaerobic biofilm was attached that conducts anaerobic degradation of organic biomass and subsequent conversion into biogas. Each of the three consecutive digesters had a total liquid volume of 10 L and 6 L of this volume was occupied by biofilm carriers.

Each of the three consecutive digesters was 20 cm wide. Each of the tubular carriers inside is 20 cm long had an open end diameter of 22 mm and an outer carrier diameter of 32 mm. The digesters were filled with liquid. Over and under the biofilm carriers were app. 5 cm free liquid. Each of the three digesters was equipped with central-shaft mounted propeller agitators in the carrier free liquid over and under the biofilm carriers. Inner diameter of the primary fluid channel defined by the tubular carriers in the absence of biofilm was 2.2 cm.

The three digesters were mounted at different vertical positions with the first digester mounted highest, the next consecutive digester 25 cm lower than the first digester and the last consecutive digester mounted 25 cm lower than the second digester. The differences in vertical mounting height allowed for liquid to flow from the first digester to the second and third by gravity.

The liquid level in all three digesters was defined by an effluent pipe above the carriers. When new feed enters the first and highest mounted digester the level in this digester will rise over the effluent pipe level and the excess liquid will leave the digester to enter the second digester which will then experience level elevation and the excess liquid from this digester will then flow to the third and last consecutive digester. From this digester, the excess liquid will flow out of the effluent pipe of the third digester into an effluent holder.

All three digesters have circulation effluent tubes in the bottom of the digester. From the effluent pipe, the digester content is continuously sucked into a peristaltic circulation pump and returned to the digester through a digester top circulation liquid inlet pipe.

The circulation flow rate was defined by the wanted flow velocity through the open diameter of the vertically oriented biofilm carriers. The circulating liquid was mixed by the propeller over the biofilm carrier before the liquid flow enters the carrier body through which the flow is a laminar plug-flow. When the circulating liquid leaves the carrier zone it was again be mixed by the agitator propeller under the carriers before repeating the circulating cycle.

Over the liquid level of the three digesters is a head-space where the produced biogas collects. The produced gas escapes the digester through a plastic tube connected to a gas flow meter with a 10 ml gas resolution. The gas production is logged in the control system.

### Internal Digester Flow Pattern

The internal circulation flow may have at least two functions:
1. Re-introduction of substrate rich liquid to the bacterial biofilm dwelling in the internal periphery of the biofilm carriers.
   By mounting the biofilm carriers with the open end of the tubes in the liquid flow direction, only periphery of the liquid flowing in a laminar plug-flow though the carriers will be in contact with the biofilm attached to the carriers. As the circulation flow velocity is too high for the substrates in the core liquid to diffuse to the bacteria dense biofilm, the substrate not in direct contact with or very close to the biofilm will not meet the fixated biogas bacteria. By mixing the liquid leaving the carrier zone and mixing the liquid entering the carrier zone, a well-mixed re-introduction is secured and during the next plug-flow passage of the carrier zone, the liquid parts that is in close proximity with the biofilm again carry undergradated substrate.
2. Counteracting debris deposition in the carrier tubes and aiding sedimenting particle transport away from the biofilm carrier zone.
   When the digester feed contains sedimenting particles and/or slowly degradable colloid particles, there is a risk that such particles will clog together to form debris and create flow obstacles slowing the liquid flow through the tubular carriers and break up the plug flow. For all biofilm to be well exploited, all tubes must be kept open to the liquid flow. If debris is allowed to settle in a tube, this tube will experience higher flow resistance than other tubes and the liquid will seize to flow through the tube as the liquid always selects the easier flow passage. This will subsequently create dead-zones in the carrier zone—and too high flow velocity in the remaining open tubes reducing the exposure time between substrate and bacteria and risking biofilm rupture by shear force.
   The sedimenting particles thus transported through the carrier zone without attaching to the biofilm carriers are allowed to sediment in the bottom of the digester and be sucked into the circulation tube along with the colloid particles to be re-introduced with the circulation stream. This in term keeps the particles suspended and enables passage of sedimenting particles through the whole system and into the effluent tank.

### Control and Operation

The system was operated automatically with pulse-pause and speed control on both feed pump and circulation pumps.

pH, digester temperature and gas flow were measured and logged on-line and could be accessed and controlled remotely.

pH, temperature and gas flows along with analysis measurements of VFA (Volatile Fatty Acids), COD (Chemical Oxygen Demand), Nitrogen and cations were used to monitor system health and provide data for test purposes.

### Example 2. Flow Tests in the Absence of Biofilm

In order to verify proper mixing, local circulation and general plug-flow distribution was measured by passing through a pulse of concentrated methylene-blue dye that could be determined with a spectrophotometer after being distributed in the system.

The RTD analysis provides a mathematical, graphical and vessel wise picture of fluid and particle distribution in the system. For optimum mixing, the total system behaves like a true plug-flow, and each digester as a CSTR notwithstanding, there are plug-flow zones in each digester. If RTD analysis shows that mixing is not optimal, it should point towards an optimal solution.

The reactors were visually inspected for proper functioning and each of them was filled with 7.5 liters of tap water. The first reactor of the cascade, was injected with a single dose of methylene blue to a final concentration of 0.0058 mM and the absorbance was recorded using a spectrophotometer set at 668 nm (wavelength where methylene blue displays maximum absorption). A constant flow of water was then introduced to the first reactor in series using a peristaltic pump and from the top, in order to have an entire volume displacement inside the reactor in a lapse of 2 hours. During this process, every 5 minutes a sample is taken from the top of each individual reactor and measured in the spectrophotometer. The RTD curve was then plotted to verify if the system has a proper mixing and if the flow occurred as intended. Results were compared with similar experiments in literature.

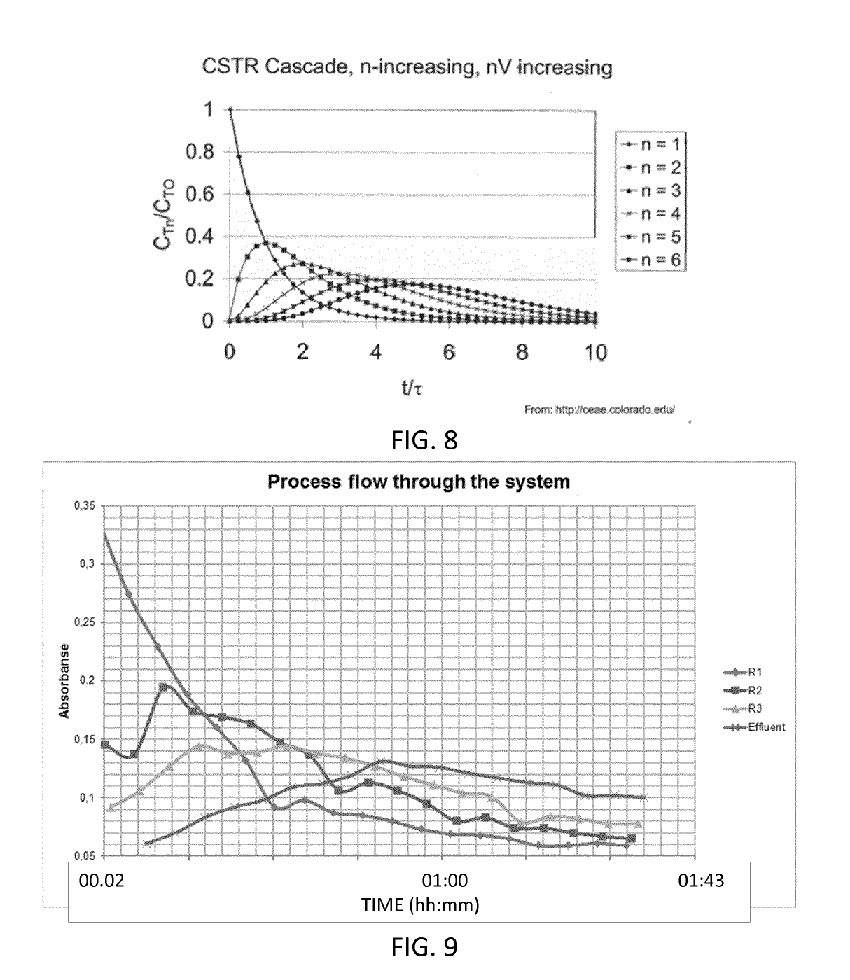

The circulation speed was set to 0.45 per Minute. The cuvettes and the spectrophotometric measurements were done soon after the sample was taken from the inside of each reactor. FIG. 8 shows an ideal behaviour of a cascade of CSTR reactors when there is proper mixing of the solution inside all the digesters. The experimental results from this RTD analysis are shown in FIG. 9. The comparison of ideal and empirical graphs showed that flow behaviour of the system was adequate. In particular, the maximum concentrations or peaks of individual digesters occurred when these overlap the previous, indicating there is a gradual increment of the total cascade volume; as expected in a well-mixed system. Furthermore, after time 60 min, the concentration of dye in all the digesters slowly decrease in the expected order and this indicates that the flow from the first to the last digester is adequate as the dye does not concentrate in any specific area of any digester of the system.

### Example 3. Digester Seeding

Many residual product biomasses contain the microbes responsible for the anaerobic biogas production from organic matter. When building up the necessary concentration of the different bacteria and Archaea bacteria the reproduction time for all microbes must be respected. In order to minimise the time consumption for the build-up of the wanted biological activity, it is recommendable to start out with a biomass that already have high concentrations of the biogas microbes.

The best match of microbial composition will be from anaerobic digesters converting a biomass similar to the biomass expected in the fully loaded FAD digester. As the FAD is expected to operate on enzymatically and microbially pre-digested (liquefied) organic fraction of municipal solid waste (MSW) and as no such digester exist, the closest are digesters operated on other types of pre-digested biomass. The Billund, Denmark, biogas digester was selected as a source of seed inoculum since this operates on source separated food waste. Most human consumables have been pre-processed and consist mostly of carbohydrates, fat and meat proteins. This was the closest match to the liquefied organic fraction of municipal solid waste (MSW) that will present the highest concentration of the required microbial consortia.

### Inoculation

Two samples of 20 L each were retrieved from the well-mixed Billund digester. The samples were transported at digester temperature to the FAD digester facility and immediately applied to the FAD digesters. As the inoculum did not fill up the digesters, warm water was added to make the correct digester level.

When taken out and transported, the methanogens can be expected to stress and become temporarily inactive. In order for the seed methanogens to acclimatise the FAD digesters were left standing for 7 days at an operation temperature of 37° C.

After the inoculation acclimatisation period had passed, a pulse shock load of 150 ml of liquefied organic fraction (LOF) of MSW (SLR of 0.6 gCOD/Vd) were injected into all digesters to test their livelihood and their readiness for beginning the load-up. The 150 ml LOF was well under the usual load per day for the digester content of the Billund Biogas digester and did not present any danger of overfeeding the inoculum bacteria. The resulting gas and a COD balance analysis indicated that the expected amount of biogas had been produced. When this digester “health” check was approved and the expected conversion rate had been shown for all the digesters, the digesters were ready for load-up.

### Example 4. Building Biofilm on High VFA Feed Stream

Before commencing the load-up of the FAD digesters, the starting load was determined. When transported, re-located, heated and diluted, the digester content cannot be expected to exert the same efficiency as it did inside the source digester. The Billund Biogas informed about the normal COD load in their digester being app. 3 gCOD/L*d, and the starting load of LOF in the FAD bench scale system was determined to be ⅔'rds (2 gCOD/L*d) for this flow to both gain a fast load-up and respect any process difficulties originating from the transfer to the FAD digesters.

The biofilm was expected to attach to the carrier and encompass the wanted microbes within a time frame of 8-12 weeks (as known in the art). The sequence will be firstly attachment of different exopolymer excreting rods and cocci followed by a diverse consortium of bacteria families over 5-7 weeks and only followed by methanogenic Archaea during the 10'th to 12'th week of the biofilm inoculation.

As the biofilm carriers were at all times covered by digester liquid, the biofilm itself could not be directly monitored. During the inoculation the digester liquid was able to convert the fed-in COD like any CSTR digester. Thus, the digester system will be loaded up in the same way a conventional biogas system is loaded up with the COD load increase respecting the growth limitation on the slowest growing microbes—the methanogens. The load-up is performed at an feed-in increase of app. 1.5% per day based on the feed-in during the preceding day.

During the conventional biogas digester load-up, the methanogen maintenance time of 10-14 days was respected. When the hydraulic load exceeds this and the hydraulic retention time (HRT) falls below 10 days, the conventional biogas digester cannot longer support the necessary reproduction of methanogenic bacteria and they will be flushed out resulting in decreasing biogas forming capacity, increasing VFA concentrations and ultimately seizure of the digester process. When the biofilm has developed correctly attached onto the biofilm carriers, the bacteria consortium in the biofilm will uphold the COD conversion efficiency even though the methanogenic concentration in the digester liquid drops. Consequently, the feed load-up to reach an HRT of 10 days takes at least 12 weeks in order to allow for the biofilm to fully develop.

When the digester COD conversion remains intact and still increases according to the COD load up, the biofilm has de-facto taken over biogas production and the continuing COD load-up shall no longer respect the reproductive speed of the methanogens in the digester liquid but that of the methanogens engulfed in, or attached to, the now formed biofilm. When the COD load-up continues to even lower HRT's it is proof that the biofilm microbial community is plastic and can be altered after the full formation in a manner that suits the purpose of degrading COD and converting it to methane gas.

The characteristics of the liquefied organic fraction of MSW used as feed for the biofilm build up are shown in Table 2. The pH was generally approximately 4.0-4.2. Total volatile fatty acids, in particular acetate and lactate, have already been fermented during enzymatic and microbial liquefaction of the organic fraction of municipal solid waste. The total and volatile solids of this LOF feed typically can oscillate between 100-120 gr solids/L and 80-100 gr/L respectively. Total solids is expressed as a percentage w/w.

TABLE 2
Characteristics of liquefied organic fraction of MSW
Feed Characteristics
Volatile fatty acids

| | | | | | | Total |
|---|---|---|---|---|---|---|
| Lactic | acetic | propionic | n-butyric | iso-valeric | n-valeric | measured |
| acid | acid | acid | acid | acid | acid | VFA |
| g/L | g/L | g/L | g/L | g/L | g/L | g/L |
| 34.17 | 8.34 | 2.55 | 2.31 | 0.04 | 0.49 | 47.91 |

Sugar Monomers and Ethanol

| Glucose | Xylose | Xylitol | Ethanol |
|---|---|---|---|
| g/L | g/L | g/L | g/L |
| 0.59 | 0.17 | 0.67 | 7.99 |

Solids and Chemical Oxygen Demand

| Total COD | Soluble COD | Total Solids | Volatile solids |
|---|---|---|---|
| g/L | g/L | % | % |
| 134.28 | 81.43 | 10.83 | 8.49 |

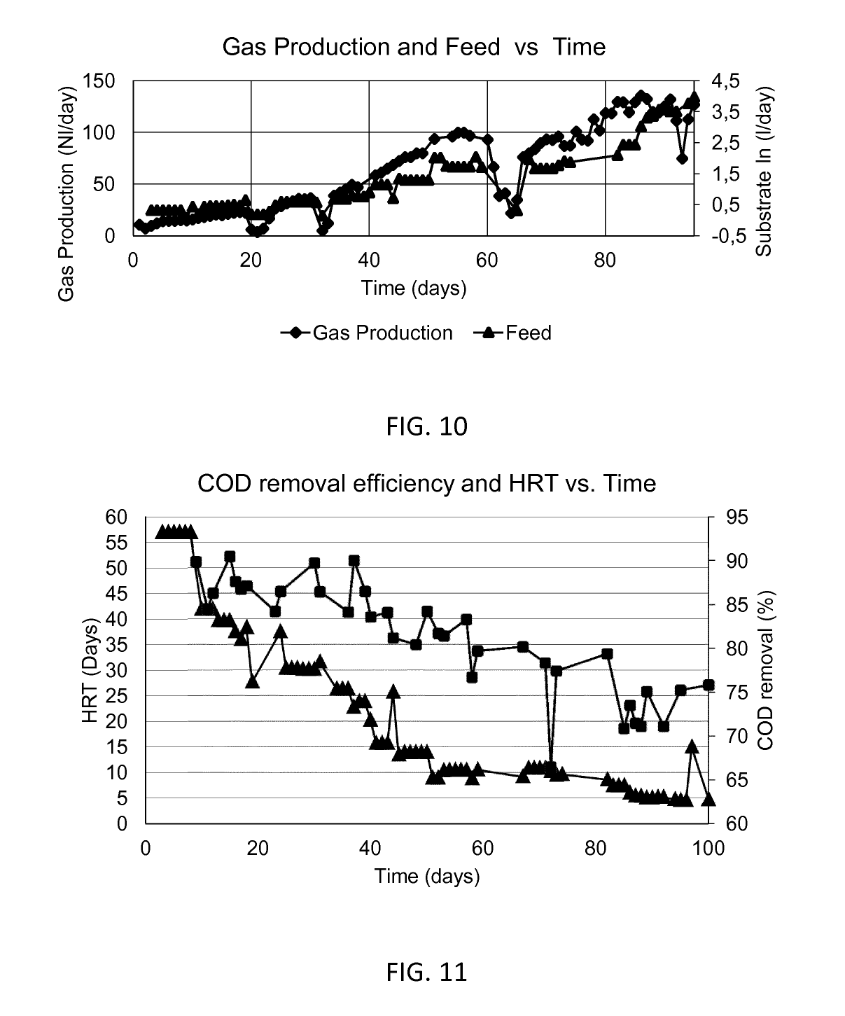

FIG. 10 shows gas production (diamonds) and feed rate (circles) in liters per day over the course of 95 days biofilm build-up of the FAD system described in example 1 using the LOF feed described in Table 2.

FIG. 11 shows COD removal in % total (squares) and hydraulic retention time (triangles) over the course of 100 days biofilm build-up of the FAD system described in example 1 using the LOF feed described in Table 2.

The COD load-up levels out when the COD conversion no longer responds by increasing the gas production when the COD load increases and the digester responds by increasing the VFA concentration in the digester effluent. In the case of the LOF feed used in biofilm buildup, this point was initially reached at 72 hours hydraulic retention time. However, this was not believed to reflect any underlying metabolic limit of the system but rather technical difficulties arising from pH balance issue using the highly acidic feed.

### Example 5. Characterization of Biofilm

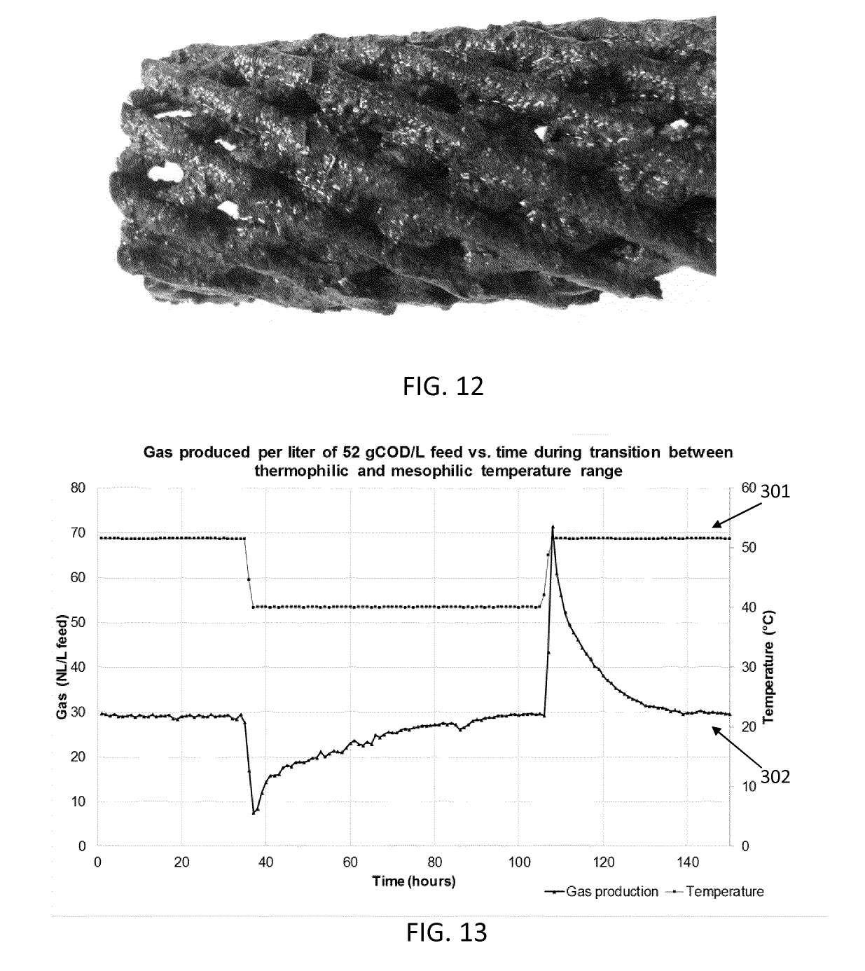

One single tube from a BIO BLOK 300™ carrier was removed from the bioreactor described in example 1 after development of biofilm as described in example 4. FIG. 12 shows a photo of the porous tubular carrier with biofilm attached. The photo was taken from the outside of the carrier where photographing was easiest. The inside of the tubular carrier looks the same. The biofilm attached to the carrier comprises a greenish, slippery material and generally exhibits a thickness of between 0.5-1.5 mm depending on whether the biofilm is growing on the thread surfaces pointing into the tube centre (thinnest biofilm) or growing in the space formed by the overlaying threads making up the carrier (thickest biofilm). The biofilm is evenly distributed over all carrier threads. The biofilm is smooth and does not exhibit any threads protruding out from its surface.

In order to perform tests on the biofilm and describe its properties “biopsies” of the carrier have been taken from the digesters. The biofilm seems to be easily removable from the carrier by means of high velocity water flushing, etc. When removing the biofilm from the carrier, the biofilm does not loosen in layers but only the film at the shear force point will rub off.

Cell density of microorganisms within a biofilm formed on a carrier can typically reach levels one order of magnitude higher than can be achieved in CSTR liquid volumes. See Langer et al. (2014). Thus it is expected that high density of methane producing Archaea within biofilm formed on porous tubular carriers in practicing methods of the invention contributes to increased biogas production performance using bioreactors of the invention. Maintaining high cell densities within the biofilm serves to protects the microbial community from process imbalances that would affect a CSTR system. Furthermore, the high organic loads used during the reactor's seeding favours the attachment of dense microbial communities to the biofilm with even higher cellular ratios than at lower organic loads.

The relative percentage of Archaea to bacteria and approximate cell densities within biofilm removed from “biopsies” as described can be determined by comparing the products from 16srRNA polymerase chain reaction (PCR) using universal 16s rRNA and Archaea-specific 16s rRNA primers reported by Gantner et al. (2011) in a DGGE gel. In so doing, it will be possible to described the Archaeal communities developed within the biofilm and to understand that ratio between them and the rest of the microorganisms responsible for breakdown of organic compounds until the production of biogas. The first digester chamber of the cascade is expected to have a higher ratio of Archaea than in the subsequent chambers since most of the easily degradable metabolized organics are converted by microbes preferring monomeric sugars, low molecular lipids, etc. in this process step. However it is possible that subsequent digester chambers comprise communities more specialized in degrading larger organic compounds that are converted into biogas. A further study regarding the quantification and determination of overall microbial groups and their distribution in the consecutive chambers can be performed to describe the above mentioned.

### Example 6. Rapid Conversion from Mesophilic to Thermophilic Conditions—Increased Conversion Rate Obviates the Need for pH Adjustment of High Solids, Acidic Feed Stream

It has previously been believed in the prior art that there exists a clear distinction between mesophilic and thermophilic Archaea producing biomethane. See e.g. Bouskova et al. (2005) and see Li et al. (2014). During the course of the experiments described in example 4, we discovered that the microbial community within the biofilm functions normally throughout the entire temperature range from 37° C. to 53° C. The difference between the temperature regimes is that COD conversion occurs faster at thermophilic temperature and—as a consequence—the need for adjusting pH of the acidic LOF feed media disappeared when temperature was from mesophilic (37° C.) to thermophilic (53° C.) conditions. The need for pH adjustment disappeared at higher temperatures because the internal buffer effect from COD conversion into CO₂, CH₄ and NH4+ occurred fast enough to counteract the acidification caused by incoming acidic feedstock.

Biofilm in the digesters was developed using LOF feed at mesophilic temperature—37° C. The temperature was subsequently raised to 52° C. over a very short period of time. This resulted in faster COD turnover and elimination of the need for pH adjustment of the acidic LOF feed. The active biology, immobilized in the biofilm, cannot change very rapidly. Ability to change process temperature up and down between mesophilic and thermophilic range in as short time as three hours indicated that the temperature flexibility is already inherent in the microbial community grown at mesophilic temperature. It is hard to imagine that thermophilic microorganisms won some selective battle during the mesophilic biofilm build-up. This indicates that the same microorganisms, when constrained within a high cell density biofilm, actually have a much greater temperature operation window than was previously believed possible. As a consequence of this previously unknown feature, bioreactors of the invention can operate in either temperature range regardless of the initial load-up temperature.

In order to document the temperature flexibility of the FAD system developed as described in examples 1, 3 and 4, the system was fed with a constant feed rate of acidic LOF feed at 53° C. until stable gas production was achieved. Temperature of the system at constant feed rate was suddenly changed to 37° C. and maintained until stable gas production was again achieved. The temperature was then restored to 53° C. and maintained until stable gas production was again achieved. The results of this experiment are shown in FIG. 13, which shows temperature (301) and gas production (302) over the course of a 145 hour run. As shown, the temperature drop to 37° C. within the digester was achieved in less than three hours. During the next 60 hours biogas production increases gradually until it reaches steady state at the same biogas production levels where it was prior to the temperature change. After production had stabilized, the temperature was again restored to thermophilic range, eventually recovering the same steady-state gas production levels. As shown, the temperature increase speeds up the process of hydrolysation, fermentation and methane formation. It is possible to observe thus, that the system can be operated at different temperature ranges without compromising the gas production and that the anaerobic process occurs faster at elevated temperatures. Rapid fluctuations that would otherwise compromise the long performance of the same process in a CSTR would consequently not be a problem for the FAD digester.

### Example 7. Long Term Stability with Minimal Requirement for Process Controls

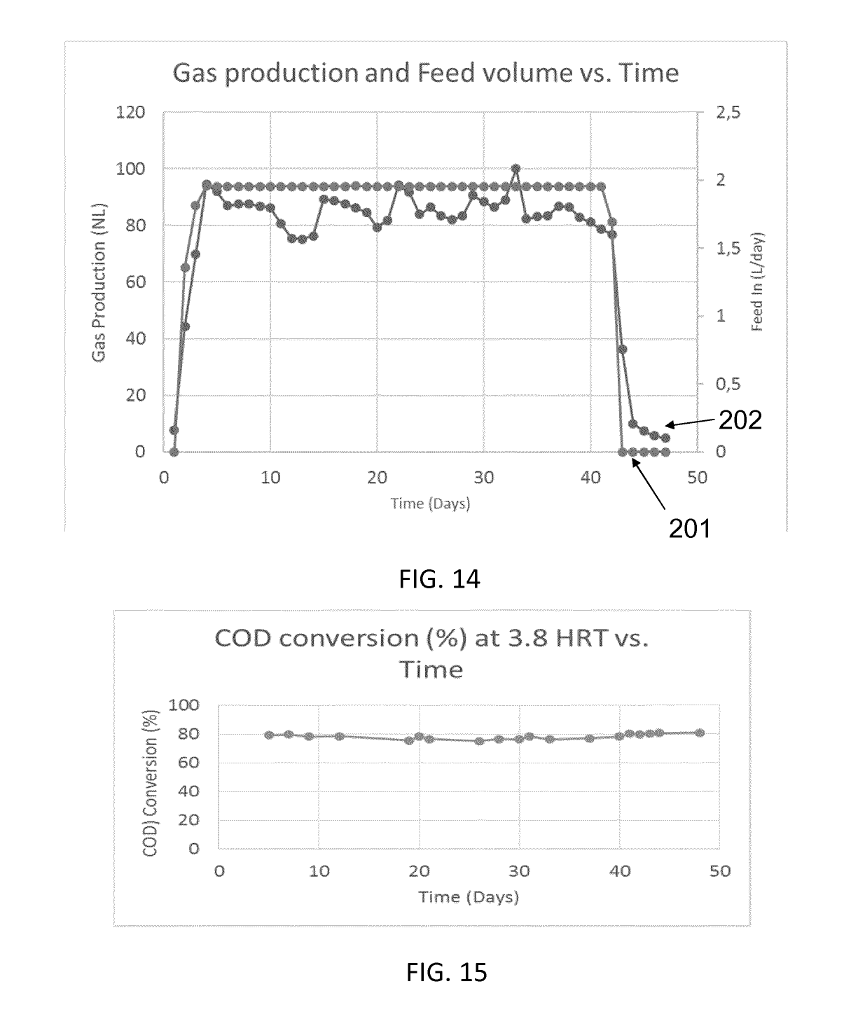

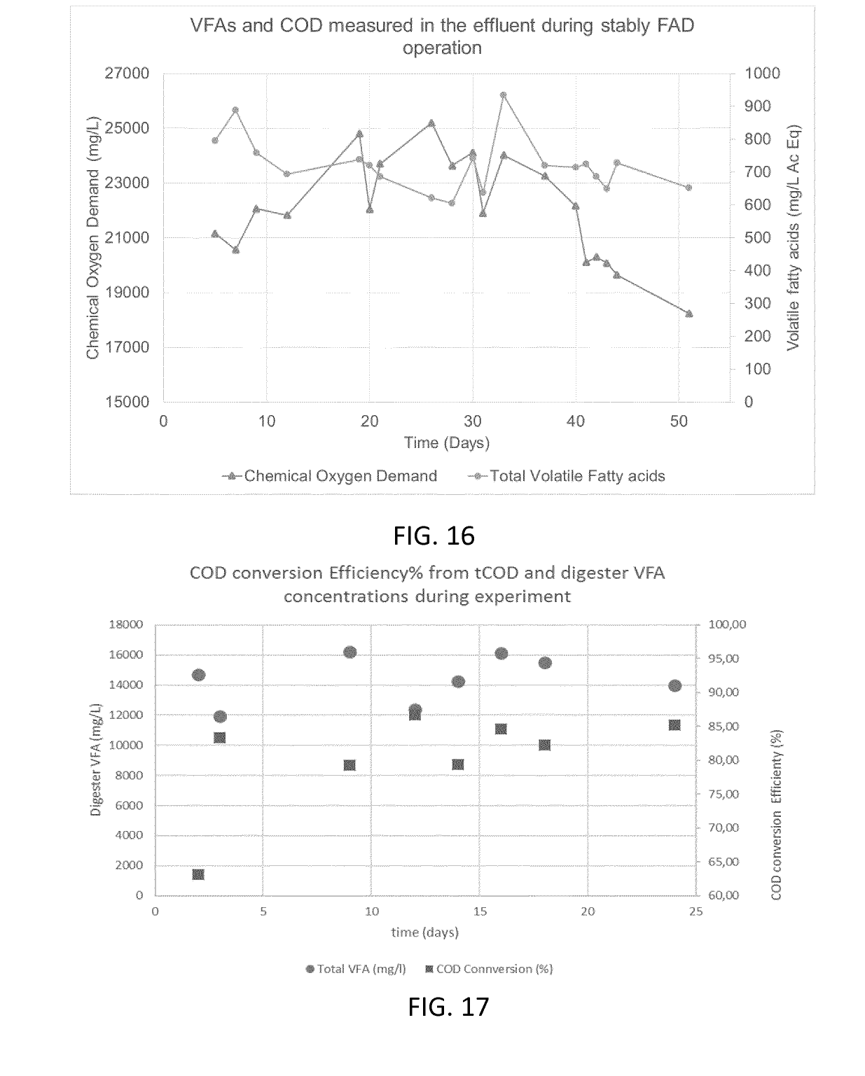

The FAD system developed as described in examples 1, 3 and 4 was fed with an LOF feedstock having the characteristics shown in Table 3 with a hydraulic retention time of 91 hours for a period of 52 days. FIG. 14 shows gas production (red circles) and feed rate (blue circles) for 48 days. FIG. 15 shows COD conversion % over a period of 48 days. FIG. 16 shows COD content (circles) and VFA levels (triangles) measured in effluent from the digester system for 51 days.

As shown, the system supports stable operation with minimal need for process controls. Such a stable operation is very beneficial in terms of the determination of both the biomass gas potential and gas production under continuous conditions. With the COD conversion efficiency preserved regardless of the feed-rate and the gas production becoming stable with the feed-in stabilises it is a very strong indicator of the real gas production under continuous conditions. In addition—still with the prerequisite that the COD conversion is unchanged—the total gas production from the first feed-in to the gas production seizes after removal of the feed will show the gas potential of the feed material just as good as if it was performed in a regular batch-test.

TABLE 3
Characteristics of REnescience C biomass for steady operation test
Feed Characteristics
Volatile fatty acids

| | | | | | | | Total |
|---|---|---|---|---|---|---|---|
| Lactic | acetic | propionic | n-butyric | iso-valeric | n-valeric | iso-Butyric | measured |
| acid | acid | acid | acid | acid | acid | acid | VFA |
| g/L | g/L | g/L | g/L | g/L | g/L | g/L | g/L |
| 28.228 | 2.518 | 0.202 | 0.072 | 0 | 0 | 0.24 | 31.26 |

Sugar Monomers and Ethanol

| Glucose | Xylose | Xylitol | Ethanol |
|---|---|---|---|
| g/L | g/L | g/L | g/L |
| 1.14 | 3.31 | 0 | 2.99 |

Solids and Chemical Oxygen Demand

| Total COD | Soluble COD | Total Nitrogen | Total Solids | Volatile solids |
|---|---|---|---|---|
| g/L | g/L | g/L | % | % |
| 134.28 | 81.43 | 1.85 | 9.43 | 7.49 |

Effluent Characteristics
Volatile fatty acids

| Acetic | Lactic | Propionic | iso-Butyric | n-Butyric | iso-Valeric | n-Valeric | Formic | Total |
|---|---|---|---|---|---|---|---|---|
| acid | acid | acid | acid | acid | acid | acid | acid | VFAs |
| g/L | g/L | g/L | g/L | g/L | g/L | g/L | g/L | g/L |
| 0.082 | 0.014 | 0.16 | 0.00 | 0 | 0.011 | 0 | 0 | 0.26 |

Sugar Monomers and Ethanol

| Cellobiose | Glucose | Xylose | Arabinose | Xylitol | Ethanol |
|---|---|---|---|---|---|
| g/L | g/L | g/L | g/L | g/L | g/L |
| 0 | 0 | 0.00 | 0.00 | 0.056 | 0 |

Solids and Chemical Oxygen Demand

| Total Solids | Volatile solids | Total COD | Soluble COD |
|---|---|---|---|
| % | % | g/L | g/L |
| 2.67 | 1.81 | 18.23 | 2.8 |

### Example 8. Immunity from VFA Toxicity

In literature, It has been described that inside an anaerobic digester, the volatile fatty acid (VFAs) concentration begins to be inhibitory over acetate concentrations of 100 mM (6 g/l) and over lower concentrations of the other VFA species (Ahring. B. K, 1994)
Reference: Ahring. B. K. (1994). Volatile fatty acids as indicators of process imbalance in anaerobic digestors. Applied Microbiology Biotechnology.

During a month period, the system described in examples 1, 3 and 4 was fed with VFA rich (38-45 g/l) and COD rich (100-130 gCOD) LOFMSW (Liquefied Organic Fraction of Municipal Solid Waste) substrate with Hydraulic retention time between 160 and 72 hours. During this period, the VFA concentration inside one digester tanks was measured to be higher than 12 g/l VFA—twice the concentration reported to be inhibitive to the biogas process. The high VFA concentration did not affect the gas production that in every case was higher than 70% total COD reduction as measured in the effluent.

FIG. 17 shows VFA concentration (circles) and conversion efficiency (squares) measured within a single digester chamber over the course of 25 days. As shown, the high VFA concentrations are clearly not inhibitory and COD conversion efficiency is not correlated with the measured VFA concentration variation. Thus, the maximal acceptable VFA concentration in the FAD system lies higher than the measured maximal concentration of 16.2 g VFA/L.

### Example 9. Immunity from Oxygen Toxicity

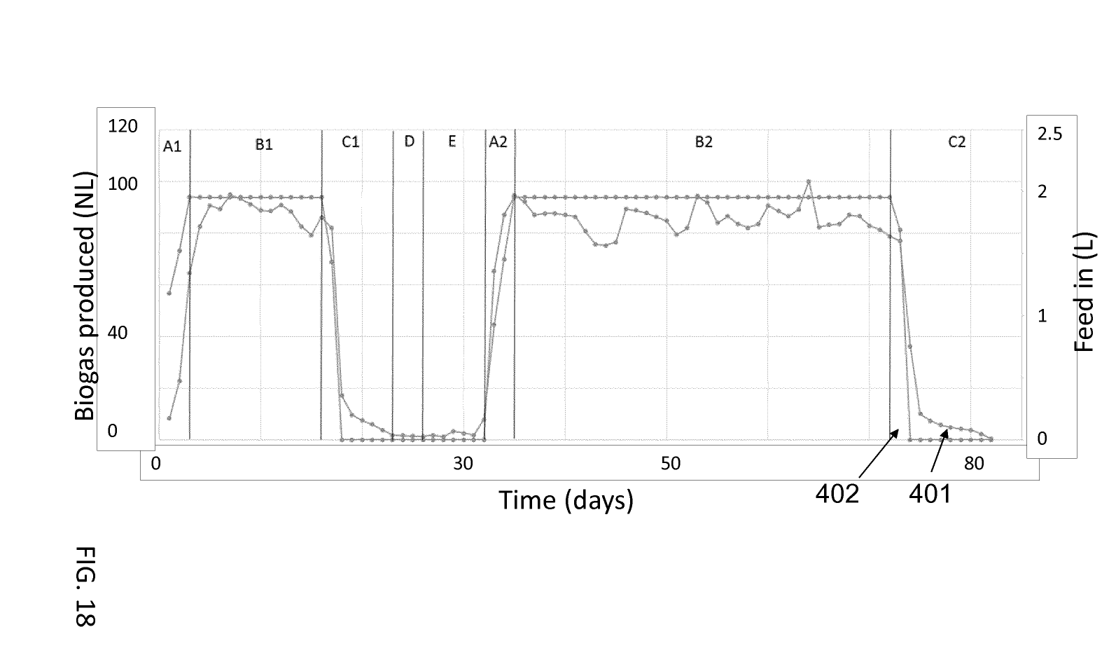

In FIG. 18 it is shown the biogas production before and after lengthy biofilm exposure to atmospheric oxygen wherein:

### A1: Load-Up Substrate 1

B1: Stable production Substrate 1

### C2: Burn-Down Substrate 1

D: Filter explosion to Oxygen
E: Rehydration of filters with effluent

### A2: Load-Up Substrate 2

B2: Stable production Substrate 2

### C2: Burn-Down Substrate 2

Line 402 shows the Feed in; line 401 the gas produced.

The system described in examples 1, 3 and 4 was fed with two similar LOF substrates; the first having a total COD load of 107.5 g COD/l and the second 101.7 g COD/l. FIG. 12 shows biogas production and feed rate during different phases of this experiment, which consist in a Load-up, Stable feed-in and gas production and Burn-down (expression of the remaining gas production after feed-in seizure) of the remaining organics of the two different substrates. Between the tests, there was a 3 day long exposure to atmospheric air of the digester immobilisation carriers, followed by a subsequent 5 day rehydration of the carriers with the effluent that had been previously evacuated from the digester. In both the scenarios, the volumetric load at the stable gas production was the same, which is 1.95 I substrate/day in the digester, corresponding to an HRT of 3.8 days.

The Load-up periods A1 and A2 consist in a two day operation in which the reactors are fed with increasing amount of substrate up to the stable production load, respectively B1 and B2. For the first substrate, the average biogas production was 88.08 NL/day, with a methane content of 61.8% for an average of 54.44 l CH4/day. This is equivalent to a 80% COD conversion efficiency.

After the first burn-down, that is C1, the liquid inside the reactor was entirely removed through the recirculation escape in the digester bottom. The digester was then flushed with an amount of tap water (at room temperature) equivalent to twice the volume of the digester. The carriers remained exposed to atmospheric oxygen for 3 days, after which the reactor was filled again with an effluent from a previous experiment, similar to that removed before the air exposure. According to the common knowledge of the fragility to air exposure the operation in D should result in a misbalance to the anaerobic digester, as it has been described that oxygen is toxic and inhibitory in conventional anaerobic digestion processes (Deshai, B 2011) Even though the exposure to the oxygen rich atmosphere should deactivate the anaerobic bacteria, this does not happen—presumably due to the protection provided by the moist biofilm. In the conventional biogas digester, inactivated bacteria leaves the digester with the effluent and is thus removing the digestion power even though the bacteria could regain activation by removal of the oxygen exposure. In the FAD digester the bacteria cannot leave and, as the latter phases clearly show, the bacteria are equally active after re-hydration and load-up of new substrate

After the rehydration (E) of the filters with effluent, an analogous load-up and stable feed in was performed in the digester. During the phase B2, the average production was 85.55 NL/day, this time with a methane content of 62% for an average of 53.04 L CH4/day. The conversion efficiency was 83%, which is very similar to the COD conversion efficiency before the exposure to oxygen. This observation shows that the system in claim is resilient to air exposure and that the continuity of the process is not compromised by this otherwise damaging operation. Furthermore, both the burn-down phases C1 and C2 were also similar. When the feed-in was stopped, the production ceased between the 5th and the 7th day. In both cases, the production had already decreased more than 50% after the first day without substrate.
(Deshai, B. (2011). Oxygen Effects in Anaerobic Digestion—A Review. The Open Waste Management Journal.

### Example 10. Feedstock Flexibility

The system described in examples 1, 3 and 4 was fed with a variety of different feedstocks.

The system in claim is flexible for operation with high gas production at high organic loading rates with different feedstock. The system has been in continuous operation at lower HTR than 5 days with dissimilar feedstock composed by different sugars, volatile fatty acids and ethanol that can be metabolically transformed in anaerobic digestion processes. The feed-in of the reactor system has been performed continuously alternating among the different feedstock and therefore different organic loading. The productivity of the digester reflects a rapid adaptation to the newly introduced feedstock as the produced biogas following the change corresponds to the potential of each feedstock that had been previously determined.

### Thin Stillage

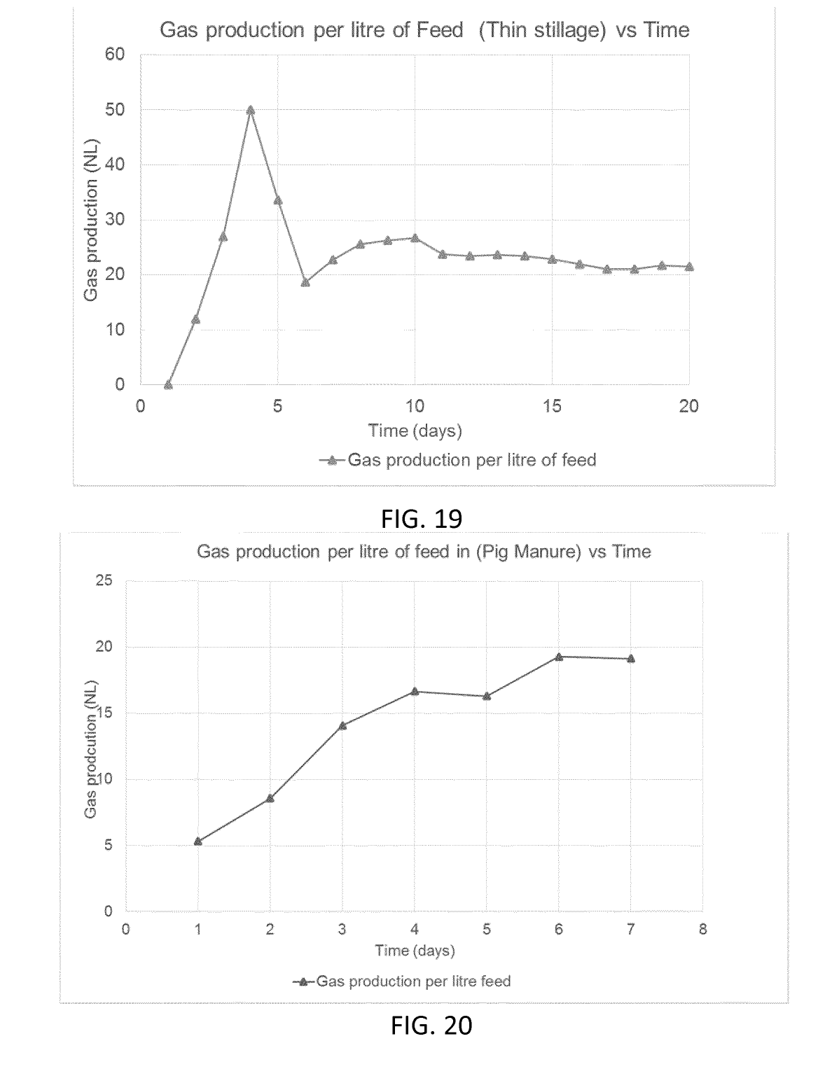

Thin stillage is a waste water fraction originating from the 2G bioethanol production. Thin stillage is free of large particles as the lignin containing particles have been separated to be used elsewhere. The thin stillage contains mainly oligomering sugars that is challenging for the biogas process as it requires a high hydrolysing power to degrade the oligomers. As it shows in FIG. 19 the FAD digester started up on LOF feed comprising predominantly monomeric sugars, fat and short lipids can easily cope with the more complex lignocellulosic Thin stillage having the characteristics shown in Table 4 comprising heavy degradable oligomeric sugars, lignin derivatives and very little lipids and proteins. Both REnescience bioliquid and Thin stillage have in common that they are pre-treated with enzymes, softening some of the harder degradable substances.

TABLE 4
Characteristics of lignocellulosic Thin stillage and Effluent produced
Feed Characteristics
Volatile fatty acids

| Acetic | Lactic | Propionic | iso-Butyric | n-Butyric | iso-Valeric | n-Valeric | Formic | Total |
|---|---|---|---|---|---|---|---|---|
| acid | acid | acid | acid | acid | acid | acid | acid | VFAs |
| g/L | g/L | g/L | g/L | g/L | g/L | g/L | g/L | g/L |
| 4.55 | 0.30 | 0.34 | 0.16 | 0 | 0 | 0 | 0 | 5.35 |

Sugar Monomers and Ethanol

| Cellobiose | Glucose | Xylose | Arabinose | Xylitol | Ethanol |
|---|---|---|---|---|---|
| g/L | g/L | g/L | g/L | g/L | g/L |
| 0.00 | 0.34 | 0.65 | 0.38 | 0.00 | 1.35 |

Solids and Chemical Oxygen Demand

| Total Solids | Volatile solids | Total COD | Soluble COD |
|---|---|---|---|
| % | % | g/L | g/L |
| 4.53 | 3.39 | 52.50 | 50.30 |

Effluent Characteristics

| Total Solids | Volatile solids | Total COD | Soluble COD |
|---|---|---|---|
| % | % | g/L | g/L |
| 2.67 | 1.81 | 6.05 | 4.09 |

### Pig Manure

The pre-treated biomasses of REnescience bioliquid from enzymated MSW and the Thin stillage form enzymated lignocellulosic biomass are both examples of biomasses that is expected to have some content of easily degradable organics that will make them good substances for the gas conversion time in a low HRT immobilised biofilm digester. In contrast, pig manure are normally thought of as a heavy degradable substance altogether as it both does not contain many easily degradables and as only app. 50% of the COD content is convertible to biogas. Consequently, it can be expected that the FAD digester will be challenged by being fed with pig manure. As can be seen in FIG. 20 the pig manure having the characteristics shown in Table 5 fed to the FAD digester performed very well, and gave the same yield as the manure would otherwise be expected to in the Maabjerg Bioenergy biogas plant from where the pig manure was sampled in the plant's feed tank. The pig manure represents a challenge to the FAD biofilm in the sense, that the biofilm microbes have been raised with the selective pressure on the fermentative and acetoclastic bacteria because the hydrolysing and acetogenic steps were already performed by the enzymatic processes preseeding the formation of the REnescience bioliquid and lignocellulosic Thin stillage. As the results from the manure test shows, the hydrolysing and acetogene bacteria are still present and active and can shift rapidly between substances. No other biogas process is known that can shift so radically and fast between so different substrates.

TABLE 5
Properties of pig manure from Maabjerg Bioenergy

| Total | Soluble |
|---|---|
| COD | COD |
| g/L | g/L |
| 59150 | 21690 |

The embodiments and examples shown are illustrative only and not intended to limit the scope of the invention as defined by the claims.

### Example “Shock Test”

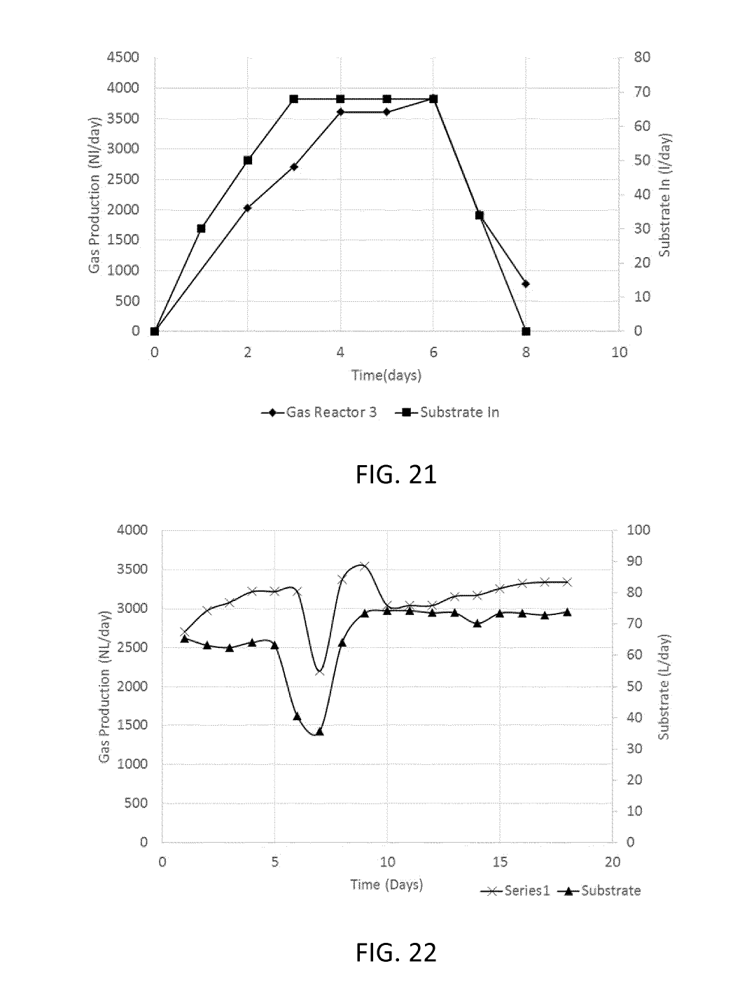

During this experiment, the feed in has been stopped for 15 days and the gas production of the 240 liter reactor was down to zero. In the lapse time of 2 days, a reactor was fed from 0 to nearly 70 liters per day, with a substrate of approximately 90 g/l of COD. The reactor was able to produce biogas without any detrimental effect due to the sudden and elevated amounts of substrate. The feed in continued for additional 5 days after this shock test. The feed in and the respective gas production during this period are depicted in FIG. 21.

This shows the ability of the bioreactor to sustain sudden stops in the feed and strong variation in the amount of feed.

### Example of Multiple Feed

This example shows the possibility of the system to produce biogas at high rates when being fed at a single and in multiple points. This feature can help to distribute the high organic loads between the compartments of the reactor. During the period of 18 days (as shown in FIG. 22), the reactor was fed in the two modalities. During the first days, all the organic load entered through the first compartment. During the next days, the substrate flow inside the reactor was distributed between the first and the third compartment. It has been proven to have high biogas production in both modalities. In any occasion during continuous operation, the substrate can be fed into any of the recirculation streams of the reactor. This could be useful, if one of the pumps fails, so the reactors operation is able to proceed.

This provide flexibility to the system as well as optimizing yield of production.

Although the present invention has been described in connection with the specified embodiments, it should not be construed as being in any way limited to the presented examples. The scope of the present invention is set out by the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

### REFERENCES

Tauseef, S. et al. “Energy recovery from wastewaters with high-rate anaerobic digesters,” Renewable and Sustainable Energy Reviews (2013) 19:704
Chen, X. et al. “Flow patterns of super-high-rate anaerobic bioreactor,” Bioresource Technology (2010) 101:7731
Mohan, S. et al. “Influence of recirculation on the performance of anaerobic sequencing batch biofilm reactor (AnSBBR) treating hypersaline composite chemical wastewater,” Bioresource Technology (2007) 98:1373
Ji, J. et al. “Hydraulic characteristics and their effects on working performance of compartmentalized anaerobic reactor,” Bioresource Technology (2012) 116:47
Alkarimiah, R. et al. “Performance of an innovative multi-stage anaerobic reactor during start-up period,” African Journal of Biotechnology (2011) 10(54):11294
Banu, J., and Kaliappan, S. “Treatment of tannery wastewater using hybrid upflow anaerobic sludge blanket reactor,” J. Environ. Eng. Sci. (2007) 6:415
Chai, S. et al. “Anaerobic treatment of winery wastewater in moving bed biofilm reactors,” Desalination and Water Treatment (2014) 52:1841
Pradeep, N. et al. “Treatment of sugar industry wastewater in anaerobic downflow stationary fixed film (DSFF) reactor,” Sugar Tech (2014) 16(1):9
Wu, S. et al. “Hydrogen production with immobilized sewage sludge in three-phase fluidized bed reactors,” Biotechnol. Prog. (2003) 19:828
Deshpande, A. et al. “Kinetic analysis of an anaerobic fixed-film fixed bed-reactor treating wastewater arising from production of a chemically synthesized pharmaceutical,” Environmental Technology (2012) 33(11):1261
Meesap, K. et al. “Microbial communities and their performances in anaerobic hybrid sludge bed-fixed film reactor for treatment of palm oil mill effluent under various organic pollutant concentrations,” Journal of Biomedicine and Biotechnology (2012) Article ID 902707
Martin, M. et al. “Kinetic evaluation of the psychrophilic anaerobic digestion of synthetic domestic sewage using an upflow filter,” Bioresource Technology (2010) 101:131
Michaud, S. et al. “Use of the methane yield to indicate the metabolic behaviour of methanogenic biofilms,” Process Biochemistry (2005) 40:2751
Austermann-Haun, U. et al. “Start-up of anaerobic fixed film reactors: Technical aspects,” Wat. Sci. Tech. (1994) 29:297
Wildenauer, F. and Winter, J. “Anaerobic digestion of high-strength acidic whey in a pH-controlled up-flow fixed film loop reactor,” Appl. Microbiol. Biotechnol. (1985) 22:367
Habouzit, F., et al. “Biofilm development during the start-up period of anaerobic biofilm reactors: the biofilm Archaea community is highly dependent on the support material,” Microbial Biotechnology (2014) 7(3):257
Adu-Gyamfi, N. et al. “Optimizing anaerobic digestion by selection of the immobilizing surface for enhanced methane production,” Bioresource Technology (2012) 120:248
Mahendran, B. et al. “Structural, physicochemical and microbial properties of flocs and biofilms in integrated fixed-film activated sludge (IFFAS) systems,” Water Research (2012) 46:5085
Hickey, R. et al. “Start-up, operation, monitoring and control of high-rate anaerobic treatment systems,” Wat. Sci. Tech. (1991) 24(8):207
Vivanco, E. et al. “Effect of addition of an exogenous exopolymeric substance in UASB and EGSB reactors,” Water Science & Technology (2006) 54(2):25
Allan, V. et al. “Development of a pH gradient within a biofilm is dependent upon the limiting nutrient,” Biotechnology Letters (1999) 21:407
Arcand, Y. et al. “Dynamic modelling of the population distribution in the anaerobic granular biofilm,” Wat. Sci. Tech. (1994) 30(12):63
Suidan, M. “Performance of deep biofilm reactors,” J. Environ. Eng. (1986) 112:78
Van Whey, A. et al. “Anisotropic nutrient transport in three-dimensional single species bacterial biofilms,” Biotechnology and Bioengineering (2012) 109(5):1280
Annachatre, A. and Khanna, P. “Fixed-film biomethanation modelling,” J. Environ. Eng. (1990) 116:49
Chen, Y. et al. “Review: Inhibition of anaerobic digestion processes,” Bioresource Technology (2008) 99:4044.
Anderson, G., and Yang, G. “pH control in anaerobic treatment of industrial wastewater,” J. Environ. Eng. (1992).
Del Pozo, R. et al. “Anaerobic pre-treatment of slaughterhouse wastewater using fixed film reactors,” Bioresource Technology (2000) 71:143.
Escudie, R. et al. “Review: Control of start-up and operation of anaerobic biofilm reactors: An overview of 15 years research,” Water Research (2011) 45:1.
Hall E. “Biomass retention and mixing characteristics in fixed-film and suspended growth anaerobic reactors.” In: Presented at IAWPR Specialised Seminar on Anaerobic Treatment, Copenhagen, Denmark, June, 1982, 1982.
Rajeshwari, K. et al. “State-of-the-art of anaerobic digestion technology for industrial wastewater treatment,” Renewable and Sustainable Energy Reviews (2000) 4:135.
Lima, C. et al. “Morphological study of biomass during the start-up period of a fixed-bed anaerobic reactor treating domestic sewage,” Brazilian Archives of Biology and Technology (2005) 48(5):841.
Albizuri, J. et al. “Validating the colloid model to optimise the design and operation of both moving-bed biofilm reactor and integrated fixed-film activated sludge systems,” Water Science & Technology (2014) 69(7):1552.
Michaud, S. et al. “Influence of hydrodynamic conditions on biofilm behaviour in a methanogenic inverse turbulent bed reactor,” Biotechnol. Prog. (2003) 19:858.
Escudie, R. et al. “Hydrodynamic and biokinetic models of an anaerobic fixed-bed reactor,” Process Biochemistry (2005) 40:2311.
Leclerc, M. et al. “Diversity of the archael community in 44 anaerobic digesters as determined by single strand conformation polymorphism analysis and 16S rDNA sequencing,” Environmental Microbiology (2004) 6(8):809.
Regueiro, L. et al. “Relationship between microbial activity and microbial community structure in six full-scale anaerobic digesters,” Microbiological Research (2012) 167:581.
Suidan, M. et al. “Optimization modelling of anaerobic biofilm reactors,” Wat. Sci. Tech. (1994) 30(12):347.
Van Loosdrecht, M. et al. “Biofilm structures,” Wat. Sci. Tech. (1995) 32(8):35.
Kennedy, K., and van den Berg, L. “Anaerobic digestion of piggery waste using a stationary fixed film reactor,” Agricultural Wastes (1982) 4:151.
Gantner, S., et al. “Novel primers for 16S rRNA-based archaeal community analyses in environmental sample,” Journal of Microbiological Methods (2011) 84:12.
Bouskova, A. et al. “Strategies for changing temperature from mesophilic to thermophilic conditions in anaerobic CSTR reactors treating sewage sludge,” Water Research (2005) 39:1481.
Li, X. et al. “Performance and microbial community profiles in an anaerobic reactor treating with simulated PTA wastewater,” Water Research (2014) 61:57.
Langer, S. et al. “Dynamics of biofilm formation during anaerobic digestion of organic waste,” Anaerobe 29 (2014) 44-51.

### Items.

1. A method of anaerobic digestion to biomethane comprising the steps of
introducing a substrate feedstock having COD content at least 30.0 g/L into a fixed film, fixed orientation, fixed bed bioreactor system in which the immobilization matrix is characterized by comprising a plurality of vertically oriented, porous tubular carriers supporting biofilm, and in which mixing zones are provided both above the upper openings and below the lower openings of the tubular carriers, and conducting anaerobic digestion of the feedstock with a hydraulic retention time of 120 hours or less while maintaining a flow velocity of at least 0.0002 m/s and a gas production rate of at least 5.0 liters/liter digester volume/day in such manner as to maintain a substantially laminar flow through the tubular carriers as well as mixing within each of said mixing zones.
   2. An anaerobic digestion bioreactor comprising a cylindrical tank having a plurality of internal, vertical biofilm carrier compartments defined by baffles or walls made from corrosion resistant and liquid impermeable material that are open at the top, where in each carrier compartment comprises a first shortened wall or underflow aperture on one side at the bottom which serves as an opening into another carrier compartment whereby fluid flows can be directed through succeeding compartments, and wherein a plurality of the carrier compartments further comprise a second shortened wall or overflow aperture at the top on a side other than that side which contains said first shortened wall or underflow aperture on one side at the bottom which serves as an opening into another carrier compartment whereby fluid flows can be directed through succeeding compartments, optionally further comprising a rotable scraper that is adapted to define sealed sections in a sedimentation zone situated beneath the lowest edge of the carrier compartments when in a closed position or to permit removal of sedimented solids when in an open position.
   3. An insert for converting a continuously stirred tank reactor (CSTR) into a fixed film, fixed orientation, fixed bed anaerobic digestion reactor said insert comprising—
interconnected baffles made from corrosion resistant and liquid impermeable material that define a plurality of vertical biofilm carrier compartments that are open at the top, each of which has a shortened wall or underflow aperture on one side at the bottom which serves as an opening into another carrier compartment whereby fluid flows can be directed through succeeding compartments, and most of which have a shortened wall or overflow aperture at the top on a side other than that which contains a shortened wall or underflow aperture at the bottom which serves as an opening into another carrier compartment whereby fluid flows can be directed through succeeding compartments.
   4. A method of converting a CSTR tank into a fixed film, fixed orientation, fixed bed anaerobic digestion reactor comprising the steps of—
assembling an insert of interconnected baffles made from corrosion resistant and liquid impermeable material that define a plurality of vertical biofilm carrier compartments that are open at the top, each of which has a shortened wall on one side at the bottom which serves as an opening into another carrier compartment whereby fluid flows can be directed through succeeding compartments, and most of which have a shortened wall at the top on a side other than that which contains a shortened wall at the bottom which serves as an opening into another carrier compartment whereby fluid flows can be directed through succeeding compartments,
installing the insert within a modified or unmodified CSTR tank,
fitting the carrier compartments defined by the insert with a plurality of porous, tubular carriers either before or after installation in the CSTR tank, and
raising a productive biofilm on the carriers.
   5. A laboratory scale device for rapid determination of biomethane potential of tested substrates adapted to practice the method of claim 1.

1. An insert comprising one or more baffles (1) defining at least two open compartments (2,3), said one or more baffles comprising one or more open edges (21), thereby when inserted into a tank reactor and when said tank reactor is in operation said one or more open edges define an underflow (22) or an overflow aperture thus forcing a fluid to flow upwardly or downwardly across said underflow or said overflow aperture.

2. An insert (14) according to claim 1, wherein said at least two compartments (6,7) further comprise a continuous closed side wall (4) surrounding said one or more baffles (5), wherein said one or more open edges (23) are displaced in respect to a height (24) of said continuous closed side wall.

3. An insert (14) according to any of the claims 1-2, wherein said one or more baffles (5) are fastened to said continuous closed side wall (4).

4. An insert (14) according to any of the claims 1-3, wherein said continuous closed side wall is a curved wall.

5. An insert (15) according to any of the claims 1-4, wherein said one or more baffles are a plurality of baffles (11,12) and wherein said at least two open compartments are a plurality of open compartments (8,9,10).

6. An insert (15) according to claim 5, wherein said one or more open edges (25, 26) of said plurality of baffles (11,12) are displaced/staggered/shifted in respect to each other, thereby when inserted into a tank reactor and when said tank reactor is in operation said one or more open edges define a plurality of underflow and overflow aperture, thus forcing a fluid to flow from an underflow aperture of a first compartment upwardly towards an overflow aperture of a second subsequent compartment and downwardly towards an underflow aperture of a third subsequent compartment.

7. An insert (20) according to any of the claims 1-6, wherein said plurality of baffles are interconnected baffles (16,17,18,19).

8. An insert (20) according to any of the claims 1-7, wherein one or more of said at least two open compartments (27, 29, 30, 31) defines one or more sections (27, 28) of said insert.

9. An insert according to claim 8, wherein said one or more sections are external (28) or internal (27) sections.

10. An insert according to any of the claims 1-9, wherein said one or more baffles and/or said continuous closed side wall are made from a corrosion resistant and liquid impermeable material.

11. An insert according to any of the claims 1-10, comprising means for supporting biofilms located in said at least two open compartments.

12. An insert according to claim 11, wherein said means for supporting biofilms are a plurality of biofilm carriers suitable for biofilm growth upon exposure to a flow of fluid containing biofilm precursors, said biofilm carrier comprising a three dimensional structure having at least one surface comprising cavities and protrusions thereby providing a rough surface.

13. An insert according to claim 12, wherein said rough surface has a rough surface area Ra between 3 and 6 mm.

14. An insert according to any of the claims 12-13, wherein said rough surface has a minimum valley Rv of 1 mm.

15. An insert according to any of the claims 12-14, wherein said rough surface has a peak depth Rp of 2 mm.

16. An insert according to any of the claims 12-15, wherein said three dimensional structure comprises openings, such as holes throughout said at least one surface.

17. An insert according to any of the claims 12-16, wherein said three dimensional structure is a tubular porous three dimensional structure.

18. An insert according to claim 17, wherein said porous are open porous.

19. An insert according to any of the preceding claims 12-18, wherein said three dimensional structure is or comprises a threaded structure.

20. An insert according to any of the preceding claims 1-19, comprising a biofilm, such as a biofilm comprising one or more different microorganisms adapted to aerobic or anaerobic digestion/fermentation.

21. An insert according to any of the preceding claims 1-20, defining a preferential vertical path along and inside said biofilm carriers, thereby when inserted into a tank reactor and when said tank reactor is in operation a fluid flow substantially parallel to the biofilm formation.

22. A bioreactor comprising:
a container having one or more side walls and a bottom wall having an internal surface and a bottom opening;
at least two removable open compartments located inside said container;
at least one overflow aperture or underflow aperture between said at least two removable compartments; thereby when in operation a fluid flows between said at least two removable compartments downwardly towards said at least one underflow aperture or upwardly towards said at least one overflow aperture.

23. A bioreactor according to claim 22, comprising said insert according to any of the claims 1-21, wherein said at least two removable open compartments are said at least two open compartments defined by said one or more baffles of said insert and wherein said at least one overflow aperture or underflow aperture are said underflow aperture or said overflow aperture defined by said one or more open edges.

24. A bioreactor according to any of the claims 22-23, wherein said one or more side walls of said container are said continuous closed side wall of an insert according to any of the claims 1-21.

25. A bioreactor according to any of the claims 22-24, further comprising, means for forcing, when in operation, a fluid to flow downwardly towards said at least one underflow aperture or upwardly towards said at least one overflow aperture through a preferential path.

26. A bioreactor according to claim 25, wherein said means for forcing a fluid to flow are said biofilm carriers according to any of the claims 12-21.

27. A bioreactor according to any of the claims 22-26, further comprising means for promoting removal of precipitate deposited or located on said internal surface of said bottom wall of said container.

28. A bioreactor according to claim 27, wherein said means for promoting removal of precipitate are one or more rotating means, such as one or more rotating scrapers.

29. A bioreactor according to claim 28, wherein each of said one or more rotating scrapers has a scraping edge and a top edge opposite to said scraping edge.

30. A bioreactor according to claim 29, wherein, when not in motion, said one or more rotating scrapers lay in a position that reduces or avoids short circuiting flow between neighbouring sections.

31. A bioreactor according to any of the claims 29-30, wherein when not in motion each of said one or more rotating scrapers rotating scrapers is located underneath a full baffle delimiting a section.

32. A bioreactor according to any of the claims 29-31, comprising a gap between said top edge of each of said one or more rotating scrapers and a edge of said full baffle, thereby ensuring for correct rotation as well as for reducing and/or avoiding cross-flow between sections.

33. A bioreactor according to any of the claims 29-32, wherein said one or more rotating scrapers are adapted to rotate clockwise or counter clockwise from a resting position underneath a first full baffle, to a second resting position underneath a second full baffle, thereby, one rotation provides scraping of the internal surface of said bottom wall of an entire section.

34. A bioreactor according to any of the claims 29-33, wherein said one or more rotating scrapers are located underneath low baffles or high baffles.

35. A bioreactor according to any of the claims 29-34, wherein said one or more rotating scrapers provide a fluid tight seal between a bottom edge of full baffles and said top edge of said one or more rotating scrapers.

36. A bioreactor according to any of the claims 23-35, wherein said container comprises a bottom chamber defined/located between said internal surface of said bottom wall and a lowest level/part of said insert according to any of the claims 1-21.

37. A bioreactor according to claim 36, wherein said bottom chamber comprises said means for promoting removal of precipitate.

38. A bioreactor according to claim 37, wherein said means for promoting removal of precipitate are adapted to define, when not in operation, static zones within said bottom chamber wherein cross-flow between compartments and said static zones is lower than a desired value, while when in operation, said static zones becomes mixing zones wherein cross-flow between compartments and said static zones is higher than said desired value.

39. A bioreactor according to any of the claims 23-38, wherein a width/size/diameter of said insert is substantially equal to a width/size/diameter of said container.

40. A bioreactor according to any of the claims 23-39, wherein said lowest level/part of said insert is located at a desired distance from said internal surface of said bottom wall.

41. A bioreactor according to any of the claims 23-40, further comprising means for keeping said insert at said desired distance from said internal surface of said bottom wall.

42. A bioreactor according to claim 41, wherein said means for keeping said insert at said desired distance are a plurality of protrusions located on said one or more side walls of said container.

43. A bioreactor according to claim 41, wherein said means for keeping said insert at said desired distance from said internal surface of said bottom wall are a curvature of said bottom wall, said curvature gradually reducing said width/size/diameter of said container, said width/size/diameter defined by said one or more side walls of said container.

44. A bioreactor according to claims 23-43, wherein when in operation, said open edges displaced in respect to each other define a plurality of underflow and overflow apertures whereby fluid flow from an underflow aperture of a first compartment upwardly towards an overflow aperture of a second subsequent compartment and downwardly towards an underflow aperture of a third subsequent compartment.

45. A bioreactor according to any of the claims 25-44, wherein when in operation, said means for forcing a fluid to flow define a preferential flow path upwardly towards an overflow aperture or downwardly towards an underflow aperture of subsequent compartments.

46. A bioreactor according to any of the claims 22-45, wherein when in operation a cross-flow in between not subsequent compartment is lower than a desired value.

47. A bioreactor according to any of the claims 22-46, further comprising means for recirculating a fluid within each of said at least two open compartments.

48. A bioreactor according to any of the claims 22-47, further comprising means for recirculating a fluid in between said at least two open compartments.

49. A bioreactor according to any of the claims 23-48, further comprising means for recirculating a fluid within each sections.

50. A bioreactor according to any of the claims 23-49, further comprising means for recirculating a fluid in between sections.

51. A bioreactor according to any of the claims 47-50, wherein said means for recirculating a fluid are one or more recirculation pumps.

52. A bioreactor according to claim 51, wherein said one or more recirculation pumps are at least in an amount equal to the amount of sections of said insert.

53. A bioreactor according to claim 51, wherein said one or more recirculation pumps are in an amount equal to the amount of compartments of said insert divided by two.

54. A bioreactor according to any of the claims 22-53, wherein said container is a cylindrical tank reactor.

55. A method of operating a bioreactor, said bioreactor according to any of the claims 22-54, said method comprising:
feeding said bioreactor with a fluid containing biofilm precursors;
conducting digestion of said fluid.

56. A method according to claim 55, wherein said fluid containing biofilm precursors is a feedstock having a COD at least 30.0 gr/L.

57. A method according to any of the claims 55-56, wherein said conducting digestion of said fluid occurs with a hydraulic retention time of 120 hours or less while maintaining a flow velocity of at least 0.0002 m/s and a gas production rate of at least 5.0 Lgas/Ldigester/day in such a manner as to maintain a substantial laminar flow through said biofilm carriers.

58. A method according to any of the claims 55-57, wherein said conducting digestion comprises forcing said fluid to flow between said at least two compartments downwardly towards said at least one underflow aperture or upwardly towards said at least overflow aperture.

59. A method according to claim 58, wherein said forcing said fluid to flow further comprises forcing said fluid to flow through a preferential flow path defined by said plurality of biofilm carriers.

60. A method according to any of the claims 58-59, wherein said forcing said fluid to flow, further comprises recirculating said fluid within each compartments.

61. A method according to any of the claims 58-60, wherein said forcing said fluid to flow, further comprises recirculating said fluid in between compartments.

62. A method according to any of the claims 58-61, wherein said forcing said fluid to flow, further comprises recirculating said fluid within each sections.

63. A method according to any of the claims 58-62, wherein said forcing said fluid to flow, further comprises recirculating said fluid in between sections.

64. A method according to any of the claims 55-63, further comprising:
removing precipitate located on said internal surface of said bottom wall of said bioreactor.

65. A system for producing biogas (74), said system comprising:
at least one feed tank (75) for feeding bioreactors;
one or more interconnected bioreactors (77) according to any of the claims 22-54;
at least one effluent tank (76) for collecting effluents from said one or more interconnected bioreactors.

66. A method of converting a Continuously Stirred tank Reactor (CSTR) having an internal surface into a fixed film, fixed orientation, fixed bed anaerobic digestion reactor, said method comprising installing an insert according to any of the claims 1-21 within said CSTR.

67. A method according to claim 66, wherein said installing comprises fastening said one or more baffles to one of more locations of said internal surface of said CSTR.

68. A method according to any of the claims 66-67, wherein said installing comprises, firstly inserting and fitting said insert in said CSTR and secondly installing said plurality of biofilm carriers.

69. A method according to any of the claims 66-68, further comprising growing a biofilm within said insert.

70. A method for performing maintenance of a CSTR modified according to the method of any of the claims 66-69, or a bioreactor according to any one of the claims 22-54, said method comprising:
temporarily interrupting a normal operation of said modified CSTR;
removing at least part of said insert;
re-installing said at least part of said insert.

71. A method according to claim 70, wherein said at least part of said insert is at least one compartment of said insert.

72. A method according to claim 70, wherein said at least part of said insert is at least one section of said insert.

73. A method according to claim 70, wherein said at least part of said insert is one or more biofilm carriers within said compartments of said insert.

74. Use of a bioreactor according to any of the claims 22-54, for producing biogas.

75. Use of a bioreactor according to any of the claims 22-54, for rapid determination of a biomethane potential of a feedstock.

76. Use of a bioreactor according to any of the claims 22-54 to produce a product produced by microbial organisms supported on a biofilm.

77. A method of aerobic or anaerobic digestion of a feedstock in the bioreactor according to any of the claims 22-54 comprising the steps of:
feeding the feedstock into the compartments of the bioreactor;
digesting the feedstock by passing the feedstock through the compartments of the bioreactor with a retention time sufficient to digest the feedstock.

78. The method of claim 77, wherein the compartments contain biofilm carriers, which have been pre-inoculated to obtain a biofilm with a suitable bacterial consortium.

79. The method of claim 78, wherein the bacterial consortium is a consortium of methane producing bacteria.

80. The method of any of the claims 77-79, wherein the feedstock is mixed between each compartment.

81. The method of any of the claim 77-79, wherein feedstock is feed simultaneously at several compartments across the bioreactor in order to form a feeding gradient.

82. The method of any of the claims 77-81, wherein the feedstock is partly or completely recirculated through the bioreactor.

83. The method of claim 82, wherein the feedstock is partly recirculated between the compartments of the bioreactor.

84. The method of any of the claims 77-83, wherein the feedstock is digested under anaerobic conditions to produce biomethane with a hydraulic retention time of 120 hours or less while maintaining a flow velocity of at least 0.0002 m/s through the bioreactor and/or a gas production rate of at least 5.0 liters/liter digester volume/day in such a manner as to maintain a substantial laminar flow through said biofilm carriers.

85. The method of any of the claims 77-84, wherein the feedstock has a chemical oxygen demand (COD) of at least 20.0 g/L, such as at least 30.0 g/L, at least 35 g/L, at least 40 g/L or at least 50 g/L or wherein the feedstock has a COD of 20-300 g/L, 30-300 g/L, 40-300 g/L, 50-300 g/L, 75-300 g/L, 100-300 g/L, such as 25-250 g/L, 30-200 g/L, 35-150 g/L, 40-150 g/L, 50-150 g/L or such as 20-125 g/L, 30-100 g/L, 30-75 g/L, 30-50 g/L, 35-75 g/L, 40-100 g/L, 50-175 g/L, 50-200 g/L.

86. The method of any of the claims 77-85, wherein the feedstock is digested at a temperature between 30 and 55° C.

87. The method of claim 86 wherein the feedstock is digested at a temperature between 37 and 48° C.

88. The method of any of claims 77-87, wherein the feedstock is digested at a pH between 6.6 and 8.5.

89. The method of claim 88, wherein the feedstock is digested at a pH between 6.8 and 7.4.

90. The method of any of claims 88 and 89, wherein the pH is adjusted by recirculation and/or addition of pH adjusting agents.

91. The method of any of claims 84-90, wherein the retention time is less than 110 hours, such as less than 100 hours, less than 90 hours, less than 80 hours, less than 75 hours, less than 60 hours, less than 50 hours, or less than 40 hours or wherein the retention time is 20-120 hours, 30-120 hours, 40-120 hours, 50-120 hours, 75-120 hours, 100-120 hours, or such as 50-110 hours, 50-100 hours, or 50-75 hours.

92. The method of any of claims 84-91, wherein the flow velocity is at least 0.00025 m/s, such as at least 0.0005 m/s, at least 0.00075 m/s, at least 0.001 m/s, at least 0.0025 m/s, at least 0.005 m/s, or at least 0.0075 m/s or wherein the flow velocity is 0.0002-0.015 m/s, such as 0.0002-0.0125 m/s, 0.0002-0.01 m/s, 0.0002-0.0075 m/s, 0.0002-0.005 m/s, or such as 0.00025-0.01 m/s, 0.0005-0.01 m/s, 0.00075-0.01 m/s, 0.001-0.01 m/s, 0.0025-0.01 m/s, 0.005-0.01 m/s, or 0.0075-0.01 m/s.

93. The method of any of claims 84-92, wherein the gas production rate is at least 6.0 liters/liter digester volume/day, such as 7.0 liters/liter digester volume/day, at least 8.0 liters/liter digester volume/day, 9.0 liters/liter digester volume/day, at least 10.0 liters/liter digester volume/day, such as at least 12.5 liters/liter digester volume/day, at least 15 liters/liter digester volume/day or at least 20 liters/liter digester volume/day wherein the gas production rate is 5.0-20 liters/liter digester volume/day, such as 6.0-20 liters/liter digester volume/day, 7.0-20 liters/liter digester volume/day 8.0-20 liters/liter digester volume/day, 9.0 liters/liter digester volume/day, or 10-20 liters/liter digester volume/day.

94. The method of any of claims 77-93, wherein the feedstock is a biomass.

95. The method of claim 94, wherein the biomass is selected from the group consisting of waste, sewage, manure, or a cellulosic, hemicellulosic, lignocellulosic or starch containing biomass selected from wheat straw, corn stover, sugar cane bagasse, sweet sorghum bagasse, distillery vinasse, or empty fruit bunches.

96. The method of claim 95, wherein the waste is selected from the group consisting of municipal solid waste (MSW), liquefied organic components of MSW, industrial waste, animal waste, plant waste or wastes from abattoirs, restaurants, dairy processing, and tanneries.

97. The method of claim 96 wherein the waste contains a level of total solids greater than 7% (w/w), such as greater than 8% (w/w), greater than 9% (w/w), greater than 10% (w/w), such as 7-20% (w/w), 8-20% (w/w), 9-20% (w/w), 10-20% (w/w), or 15-20% (w/w).

98. The method of any of claims 94-97, wherein the biomass has been subjected at least in part to one or more of hydrothermal pre-treatment, enzymatic hydrolysis and/or aerobic digestion.

99. The method according to any of the claims 84-98, wherein the feedstock is digested under anaerobic conditions to produce biomethane with a hydraulic retention time of 120 hours, or less, such as less than 110 hours, such as less than 100 hours, less than 90 hours, less than 80 hours, less than 75 hours, less than 60 hours, less than 50 hours, or less than 40 hours or wherein the retention time is 20-120 hours, 30-120 hours, 40-120 hours, 50-120 hours, 75-120 hours, 100-120 hours, or such as 50-110 hours, 50-100 hours, 50-75 hours, while maintaining a flow velocity of at least 0.0002 m/s through the bioreactor and/or a gas production rate of at least 5.0 liters/liter digester volume/day in such a manner as to maintain a substantial laminar flow through said biofilm carriers.

100. The method according to any of the claims 84-99, wherein the feedstock is digested under anaerobic conditions to produce biomethane with a hydraulic retention time of 120 hours, or less, while maintaining a flow velocity of at least 0.0002 m/s, such as a flow velocity between 0.0002 m/s to 0.08 m/s, such as between 0.0030 and 0.07, such as between 0.009 and 0.05, such as between 0.015 m/s to 0.045 m/s through the bioreactor and/or a gas production rate of at least 5.0 liters/liter digester volume/day in such a manner as to maintain a substantial laminar flow through said biofilm carriers.

101. The method according to any of the claims 84-100, wherein the feedstock is digested under anaerobic conditions to produce biomethane with a hydraulic retention time of 120 hours, or less, while maintaining a flow velocity of at least 0.0002 m/s through the bioreactor and/or a gas production rate of at least 5.0 liters/liter digester volume/day (L/L/D), such as in between 6.0 L/L/D and 10.0 L/L/D, such as in between 7.0 L/L/D and 9.0 L/L/D, such as at least 8.0 L/L/D.
